(19) United States
(12) Patent Application Publication
(10) Pub. No.: US 2024/0350680 A1
(43) Pub. Date: Oct. 24, 2024

(54) LABELED INHIBITORS OF PROSTATE SPECIFIC MEMBRANE ANTIGEN (PSMA) AS AGENTS FOR THE TREATMENT OF PROSTATE CANCER

(71) Applicant: NOVARTIS AG, BASEL (CH)

(72) Inventors: Matthias EDER, Mannheim (DE); Klaus KOPKA, Dossenheim (DE); Martin SCHÄFER, Neckarsteinach (DE); Ulrike BAUDER-WÜST, Schriesheim (DE); Michael EISENHUT, Heidelberg (DE); Walter MIER, Bensheim (DE); Martina BENESOVA, Heidelberg (DE)

(21) Appl. No.: 18/440,783

(22) Filed: Feb. 13, 2024

Related U.S. Application Data
(62) Division of application No. 17/110,558, filed on Dec. 3, 2020, now Pat. No. 11,931,430.
Application No. 17/110,558 is a division of application No. 16/551,198, filed on Aug. 26, 2019, now Pat. No. 11,045,564.
Application No. 16/551,198 is a division of application No. 16/114,988, filed on Aug. 28, 2018, now Pat. No. 10,398,791.
Application No. 16/114,988 is a continuation of application No. 15/131,118, filed on Apr. 18, 2016, now abandoned.
Application No. 15/131,118 is a continuation-in-part of application No. PCT/EP2014/002808, filed on Oct. 17, 2014.

(30) Foreign Application Priority Data
Oct. 18, 2013 (EP) 13004991.9
Jul. 3, 2014 (EP) 14175612.2

(51) Int. Cl. A61K 51/04; C07B 59/00; C07D 257/02; C07D 295/145

(52) U.S. Cl. CPC A61K 51/0482; A61K 51/0402; A61K 51/0497; C07B 59/002; C07D 257/02; C07D 295/145

(57) ABSTRACT

The present invention generally relates to the field of radiopharmaceuticals and their use in nuclear medicine as tracers, imaging agents and for the treatment of various disease states of prostate cancer. Thus, the present invention concerns compounds that are represented by the general Formulae (Ia) or (Ib).

### RELATED APPLICATIONS AND INCORPORATION BY REFERENCE

This application is a continuation-in-part application of international patent application Serial No. PCT/EP2014/002808 filed Oct. 17, 2014, which published as PCT Publication No. WO 2015/055318 on Apr. 23, 2015, which claims benefit of European patent application Serial Nos. 13004991.9 filed Oct. 18, 2013 and 14175612.2 filed Jul. 3, 2014.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention generally relates to the field of radiopharmaceuticals and their use in nuclear medicine as tracers, imaging agents and for the treatment of various disease states of prostate cancer.

### BACKGROUND OF THE INVENTION

Prostate cancer (PCa) is the leading cancer in the US and European population. At least 1-2 million men in the western hemisphere suffer from prostate cancer and it is estimated that the disease will strike one in six men between the ages of 55 and 85. There are more than 300.000 new cases of prostate cancer diagnosed each year in USA. The mortality from the disease is second only to lung cancer. Currently anatomic methods, such as computed tomography (CT), magnetic resonance (MR) imaging and ultrasound, predominate for clinical imaging of prostate cancer. An estimated $2 billion is currently spent worldwide on surgical, radiation, drug therapy and minimally invasive treatments. However, there is presently no effective therapy for relapsing, metastatic, androgen-independent prostate cancer.

A variety of experimental low molecular weight PCa imaging agents are currently being pursued clinically, including radiolabeled choline analogs [¹⁸F]fluorodihydrotestosterone ([¹⁸F]FDHT), anti-1-amino-3-[¹⁸F]fluorocyclobutyl-1-carboxylic acid (anti[18F]F-FACBC, [¹¹C]acetate and 1-(2-deoxy-2-[¹⁸F]flouro-L-arabinofuranosyl)-5-methyluracil ([¹⁸F]FMAU) (Scher, B.; et al. Eur J Nucl Med Mol Imaging 2007, 34, 45-53; Rinnab, L.; et al. BJU Int 2007, 100, 786,793; Reske, S. N.; et al. J Nucl Med 2006, 47, 1249-1254; Zophel, K.; Kotzerke, J. Eur J Nucl Med Mol Imaging 2004, 31, 756-759; Vees, H.; et al. BJU Int 2007, 99, 1415-1420; Larson, S. M.; et al. J Nucl Med 2004, 45, 366-373; Schuster, D. M.; et al. J Nucl Med 2007, 48, 56-63, Tehrani, O. S.; et al. J Nucl Med 2007, 48, 1436-1441). Each operates by a different mechanism and has certain advantages, e.g., low urinary excretion for [¹¹C]choline, and disadvantages, such as the short physical half-life of positron-emitting radionuclides.

It is well known that tumors may express unique proteins associated with their malignant phenotype or may over-express normal constituent proteins in greater number than normal cells. The expression of distinct proteins on the surface of tumor cells offers the opportunity to diagnose and characterize disease by probing the phenotypic identity and biochemical composition and activity of the tumor. Radioactive molecules that selectively bind to specific tumor cell surface proteins provide an attractive route for imaging and treating tumors under non-invasive conditions. A promising new series of low molecular weight imaging agents targets the prostate-specific membrane antigen (PSMA) (Mease R. C. et al. Clin Cancer Res. 2008, 14, 3036-3043; Foss, C. A.; et al. Clin Cancer Res 2005, 11, 4022-4028; Pomper, M. G.; et al. Mol Imaging 2002, 1, 96-101; Zhou, J.; etr al. Nat Rev Drug Discov 2005, 4, 1015-1026; WO 2013/022797).

PSMA is a trans-membrane, 750 amino acid type II glycoprotein that has abundant and restricted expression on the surface of PCa, particularly in androgen-independent, advanced and metastatic disease (Schulke, N., et al. Proc Natl Acad Sci USA 2003, 100, 12590-12595). The latter is important since almost all PCa become androgen independent over the time. PSMA possesses the criteria of a promising target for therapy, i.e., abundant and restricted (to prostate) expression at all stages of the disease, presentation at the cell surface but not shed into the circulation and association with enzymatic or signaling activity (Schulke, N.; et al. Proc. Natl. Acad. Sci. USA 2003, 100, 12590-12595). The PSMA gene is located on the short arm of chromosome 11 and functions both as a folate hydrolase and neuropeptidase. It has neuropeptidase function that is equivalent to glutamate carboxypeptidase II (GCPII), which is referred to as the “brain PSMA”, and may modulate glutamatergic transmission by cleaving N-acetylaspartylglutamate (NAAG) to N-acetylaspartate (NAA) and glutamate (Nan, F.; et al. J Med Chem 2000, 43, 772-774). There are up to 10⁶ PSMA molecules per cancer cell, further suggesting it as an ideal target for imaging and therapy with radionuclide-based techniques (Tasch, J.; et al. Crit Rev Immunol 2001, 21, 249-261).

The radio-immunoconjugate of the anti-PSMA monoclonal antibody (mAb) 7E11, known as the PROSTASCINT® scan, is currently being used to diagnose prostate cancer metastasis and recurrence. However, this agent tends to produce images that are challenging to interpret (Lange, P. H. PROSTASCINT scan for staging prostate cancer. Urology 2001, 57, 402-406; Haseman, M. K.; et al. Cancer Biother Radiopharm 2000, 15, 131-140; Rosenthal, S. A.; et al. Tech Urol 2001, 7, 27-37). More recently, monoclonal antibodies have been developed that bind to the extracellular domain of PSMA and have been radiolabeled and shown to accumulate in PSMA-positive prostate tumor models in animals. However, diagnosis and tumor detection using monoclonal antibodies has been limited by the low permeability of the monoclonal antibody in solid tumors.

The selective targeting of cancer cells with radiopharmaceuticals, either for imaging or therapeutic purposes is challenging. A variety of radionuclides are known to be useful for radio-imaging or cancer radiotherapy, including ¹¹¹In, ⁹⁰Y, ⁶⁸Ga, ¹⁷⁷Lu, ^{99m}Tc, ¹²³I and ¹³¹I. Recently it has been shown that some compounds containing a glutamate-urea-glutamate (GUG) or a glutamate-urea-lysine (GUL) recognition element linked to a radionuclide-ligand conjugate exhibit high affinity for PSMA.

Citation or identification of any document in this application is not an admission that such document is available as prior art to the present invention.

### SUMMARY OF THE INVENTION

New agents that will enable rapid visualization of prostate cancer and specific targeting to allow radiotherapy present are needed.

Thus, the object of the present invention is to develop ligands that interact with PSMA and carry appropriate radionuclides which provide a promising and novel targeting option for the detection, treatment and management of prostate cancer.

The solution of said object is achieved by providing the embodiments characterized in the claims.

The inventors found new compounds which are useful radiopharmaceuticals and their use in nuclear medicine as tracers, imaging agents and for the treatment of various disease states of prostate cancer.

The novel imaging agents with structural modifications in the linker region have improved tumor targeting properties and pharmacokinetics. The pharmacophore presents three carboxylic groups able to interact with the respective side chains of PSMA and an oxygen as part of zinc complexation in the active center. Besides these obligatory interactions, the inventors were able to optimize the lipophilic interactions in the linker region.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53 (c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIG. 1: PET—Imaging of MB17. Whole-body coronal microPET images of an athymic male nude mice bearing LNCaP tumor xenografts. The tumor-targeting efficacy and pharmacokinetic properties of [⁶⁸Ga]MB17 were evaluated by dynamic microPET scans. Approximately 15 MBq/mouse were injected. Graph A shows the respective time-activity-curves of kidney and bladder and graph B the respective time-activity-curves of heart, muscle and tumor. The values are expressed as mean SUV (standardized uptake values).

FIG. 2: Organ Distribution at 1 h post injection. Organ distribution at one hour post injection of 0.06 nmol of the ⁶⁸Ga labeled PSMA inhibitor MB17. PSMA-blocking by co-administration of 2 mg/kg body weight 2-PMPA indicates PSMA-specific uptake in the tumor and the kidneys. Data are expressed as mean % ID/g tissue±SD (n=3).

FIG. 3: PET-Imaging of MB4. Whole-body coronal microPET images of an athymic male nude mice bearing LNCaP tumor xenografts. The tumor-targeting efficacy and pharmacokinetic properties of [⁶⁸Ga]MB4 were evaluated by dynamic microPET scans. Approximately 15 MBq/mouse were injected. Graph A shows the respective time-activity-curves of kidney and bladder and graph B the respective time-activity-curves of heart, muscle and tumor. The values are expressed as mean SUV (standardized uptake values)

FIG. 4: Organ distribution expressed as % ID/g tissue±SD (n=5) 24 h post injection of 0.06 nmol of the ¹⁷⁷Lu-labeled MB17. Organ distribution with ¹⁷⁷Lu shows that the high initial kidney uptake is nearly completely washed out (2.13±1.36% ID/g) after 24 hours while the tumor uptake remained high and even increased (10.58±4.50% ID/g). Other organs as liver (0.08±0.03% ID/g), lung (0.11±0.13% ID/g) and spleen (0.13±0.05% ID/g) showed very low uptake. The favourable pharmacokinetics led to extremely high tumor-to-background ratios (Tumor/Blood: 1058; Tumor/Muscle: 529) after 24 hours

FIG. 5: PET—Imaging of MB 2. Whole-body coronal microPET images of an athymic male nude mouse bearing LNCaP tumor xenografts. The tumor-targeting efficacy and pharmacokinetic properties of [⁶⁸Ga]MB2 were evaluated by dynamic microPET scans. Approximately 15 MBq/mouse were injected.

FIG. 6: PET-Imaging of MB 3. Whole-body coronal microPET images of an athymic male nude mouse bearing LNCaP tumor xenografts. The tumor-targeting efficacy and pharmacokinetic properties of [⁶⁸Ga]MB 3 were evaluated by dynamic microPET scans Approximately 15 MBq/mouse were injected.

FIG. 7: PET-Imaging of MB10. Whole-body coronal microPET images of an athymic male nude mouse bearing LNCaP tumor xenografts. The tumor-targeting efficacy and pharmacokinetic properties of [⁶⁸Ga]MB10 were evaluated by dynamic microPET scans. Approximately 15 MBq/mouse were injected.

FIG. 8: PET-Imaging of MB17.D. Whole-body coronal microPET images of an athymic male nude mouse bearing LNCaP tumor xenografts. The tumor-targeting efficacy and pharmacokinetic properties of [⁶⁸Ga]MB17.D were evaluated by dynamic microPET scans. Approximately 15 MBq/mouse were injected. MB17D: stereoisomer of MB17(L), synthesis based on Fmoc-3 (2-naphthyl)-D-alanine

FIG. 9: PET-Imaging of MB22. Whole-body coronal microPET images of an athymic male nude mouse bearing LNCaP tumor xenografts. The tumor-targeting efficacy and pharmacokinetic properties of [⁶⁸Ga]MB22 were evaluated by dynamic microPET scans. Approximately 15 MBq/mouse were injected.

FIG. 10: PET-Imaging of MB 24. Whole-body coronal microPET images of an athymic male nude mouse bearing LNCaP tumor xenografts. The tumor-targeting efficacy and pharmacokinetic properties of [⁶⁸Ga]MB 24 were evaluated by dynamic microPET scans. Approximately 15 MBq/mouse were injected.

FIG. 11: PET-Imaging of MB25. Whole-body coronal microPET images of an athymic male nude mouse bearing LNCaP tumor xenografts. The tumor-targeting efficacy and pharmacokinetic properties of [⁶⁸Ga]MB25 were evaluated by dynamic microPET scans. Approximately 15 MBq/mouse were injected.

FIG. 12: PET-Imaging of MB31. Whole-body coronal microPET images of an athymic male nude mouse bearing LNCaP tumor xenografts. The tumor-targeting efficacy and pharmacokinetic properties of [⁶⁸Ga]MB31 were evaluated by dynamic microPET scans. Approximately 15 MBq/mouse were injected.

FIG. 13: PET-Imaging of MB33. Whole-body coronal microPET images of an athymic male nude mouse bearing LNCaP tumor xenografts. The tumor-targeting efficacy and pharmacokinetic properties of [⁶⁸Ga]MB33 were evaluated by dynamic microPET scans. Approximately 15 MBq/mouse were injected.

FIG. 14: PET-Imaging of MB35. Whole-body coronal microPET images of an athymic male nude mouse bearing LNCaP tumor xenografts. The tumor-targeting efficacy and pharmacokinetic properties of [⁶⁸Ga]MB35 were evaluated by dynamic microPET scans. Approximately 15 MBq/mouse were injected.

FIG. 15: PET scan of a mouse injected with ⁶⁸Ga-CHX-DTPA. On the left the caudal, in the centre the dorsal and on the right the lateral view. The pictures cover the time spans of 20-40 min (top), 40-60 min (centre) and 120-140 min (bottom).

FIG. 16: MB-17 vs MB-17.D. Whole-body coronal microPET images of athymic male nude mice bearing LNCaP tumor xenografts. The tumor-targeting efficacy and pharmacokinetic properties of the stereoisomers MB-17 and MB-17 D were directly compared at 2 hours post injection.

FIGS. 17A-B: Human PET/CT imaging 68 Ga-labeled MB17. (a) First clinical experience with ⁶⁸Ga-labeled MB17 PET/CT demonstrates the detection of small lymph node metastases 1 hour post injection, primarily due to a high radiotracer uptake. Red arrows point to a representative lesion with a SUVmax of 36.5 and a tumor-to-background ratio of 52.1 one hour post injection. MIP=maximum intensity projection of the PET 1 h post injection. (b) The significant advantage of ⁶⁸Ga-labeled MB17 PET/CT is the sensitive detection of lesions even at low PSA level.

FIGS. 18A-B: PET imaging of patient with multiple prostate cancer metastasis. (a) First scan demonstrate initial PET imaging of the patient with multiple prostate cancer metastases with blood PSA value of 14. Two months later 3.3 GBq of ¹⁷⁷Lu-labeled MB17 was applied. At this time point, the amount of PSA in blood reached a value of 38. After the first cycle, the PSA level decreased to 8. Three months after the first cycle another 4 GBq of ¹⁷⁷Lu-labeled MB17 was applied. The control PET scan was performed one month after the second cycle. The treatment has shown a significant impact on the tumor lesions and PSA value and resulted in a reduction of bone pain. (b) The graph demonstrates the significant impact on the PSA value which decreased after the first application of the therapeutic dose of ¹⁷⁷Lu-labeled MB17.

### DETAILED DESCRIPTION OF THE INVENTION

The present invention relates to radiopharmaceuticals and their use in nuclear medicine as tracers, imaging agents and for the treatment of various disease states of prostate cancer.

Thus, the present invention concerns compounds that are represented by the general Formulae (Ia) or (Ib):

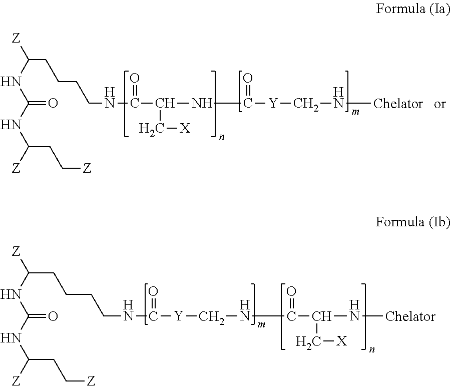

with:

| | |
|---|---|
| n: | 0, 1 |
| m: | 1, 2, 3, 4 |
| Z: | —CO₂H, —SO₂H, —SO₃H, —SO₄H, —PO₂H, —PO₃H, —PO₄H₂ |
| X: | Naphthyl, Phenyl, Biphenyl, Indolyl (=2,3-benzopyrrolyl), Benzothiazolyl |
| Y: | Aryl, Alkylaryl, Cyclopentyl, Cyclohexyl, Cycloheptyl |
| Chelator | 1,4,7,10-tetraazacyclododecane-N,N′,N″,N″′-tetraacetic acid (=DOTA), |
| radical of: | N,N″-bis[2-hydroxy-5-(carboxyethyl)benzyl]ethylenediamine-N,N″- |
| | diacetic acid (HBED-CC), |
| | 1,4,7-triazacyclononane-1,4,7-triacetic acid (=NOTA), |
| | 2-(4,7-bis(carboxymethyl)-1,4,7-triazonan-1-yl)pentanedioic acid |
| | (NODAGA), |
| | 2-(4,7,10-tris(carboxymethyl)-1,4,7,10-tetraazacyclododecan-1- |
| | yl)pentanedioic acid |
| | (DOTAGA), |
| | 1,4,7-triazacyclononane phosphinic acid (TRAP), |
| | 1,4,7-triazacyclononane-1-[methyl(2-carboxyethyl)phosphinic acid]-4,7- |
| | bis[methyl(2-hydroxymethyl)phosphinic acid] (NOPO), |
| | 3,6,9,15-tetraazabicyclo[9.3.1]pentadeca-1(15),11,13-triene-3,6,9-triacetic |
| | acid (PCTA), |
| | N′-{5-[Acetyl(hydroxy)amino] pentyl}-N-[5-({4-[(5- |
| | aminopentyl)(hydroxy)amino]-4-oxobutanoyl}amino)pentyl]-N- |
| | hydroxysuccinamide (DFO), |
| | Diethylenetriaminepentaacetic acid (DTPA) |
| | Trans-cyclohexyl-diethylenetriaminepentaacetic acid (CHX-DTPA) |
| | 1-oxa-4,7,10-triazacyclododecane-4,7,10-triacetic acid (oxo-Do3A) |
| | p-isothiocyanatobenzyl-DTPA (SCN-Bz-DTPA) |
| | 1-(p-isothiocyanatobenzyl)-3-methyl-DTPA (1B3M) |
| | 2-(p-isothiocyanatobenzyl)-4-methyl-DTPA (1M3B) |
| | 1-(2)-methyl-4-isocyanatobenzyl-DTPA (MX-DTPA) |

If not stated otherwise, in the present invention the “alkyl” residue (preferably: C₁ to C₁₀) can be linear or branched, unsubstituted or substituted. Preferred alkyl residues are methyl, ethyl, n-propyl, iso-propyl, n-butyl, tert-butyl, n-pentanyl, n-hexanyl. The same also applies to the corresponding cycloalkyl compounds having preferably 3 to 10 carbon atoms.

“Aryl” refers to an aromatic monocyclic or polycyclic ring system having 6 to 14 carbon atoms, preferably 6 to 10 carbon atoms. The aryl group can be substituted, where appropriate, with one or several ring substituents, like alkyl groups. Preferred aryl groups are phenyl, benzyl or naphthyl.

Although it is preferred that the Z-Group is —CO₂H it may be easily replaced with biosteric replacements such as —SO₂H, —SO₃H, —SO₄H, —PO₂H, —PO₃H, —PO₄H₂, see e.g. “The Practice of Medicinal Chemistry” (Academic Press New York, 1996), page 203.

Within the meaning of the invention, all residues are considered combinable unless stated otherwise in the definition of the residues. All conceivable subgroupings thereof are considered to be disclosed.

In a preferred embodiment, the motif specifically binding to cell membranes of neoplastic cells is a motif specifically binding to cell membranes of cancerous cells, preferably wherein said motif may comprise a prostate-specific membrane antigen (PSMA), in particular wherein said PSMA may comprise a glutamate-urea-lysine motif according to the following formula in Scheme 1.

Thus, preferred molecules of the present invention consist of three principle components (Scheme 1): the hydrophilic PSMA binding motif (Glu-Urea-Lys; =Glu-NH—CO—NH-Lys), a variable linker and the chelator which is preferably DOTA.

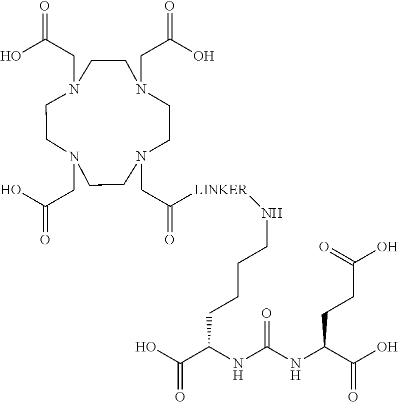

The different preferred linkers are shown below, wherein R=Glu-urea-Lys and R′=DOTA (as a preferred example for the chelator), as shown above.

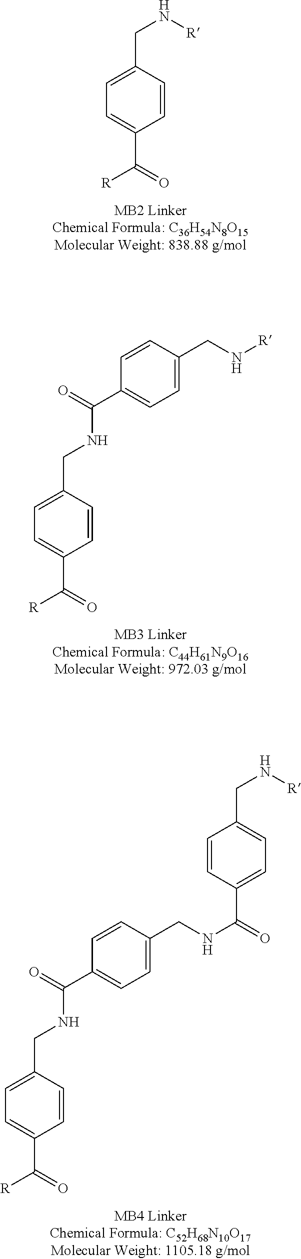

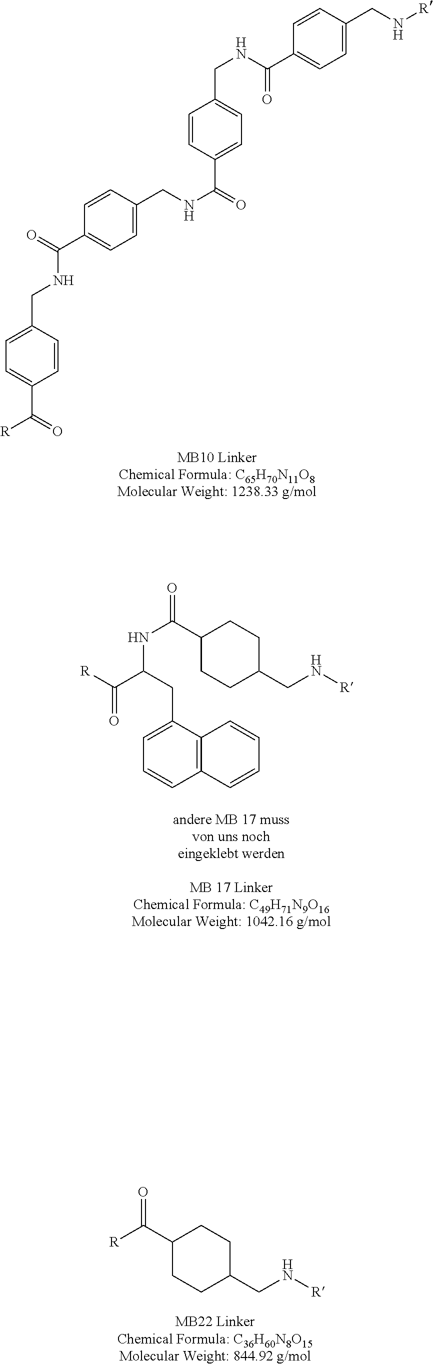

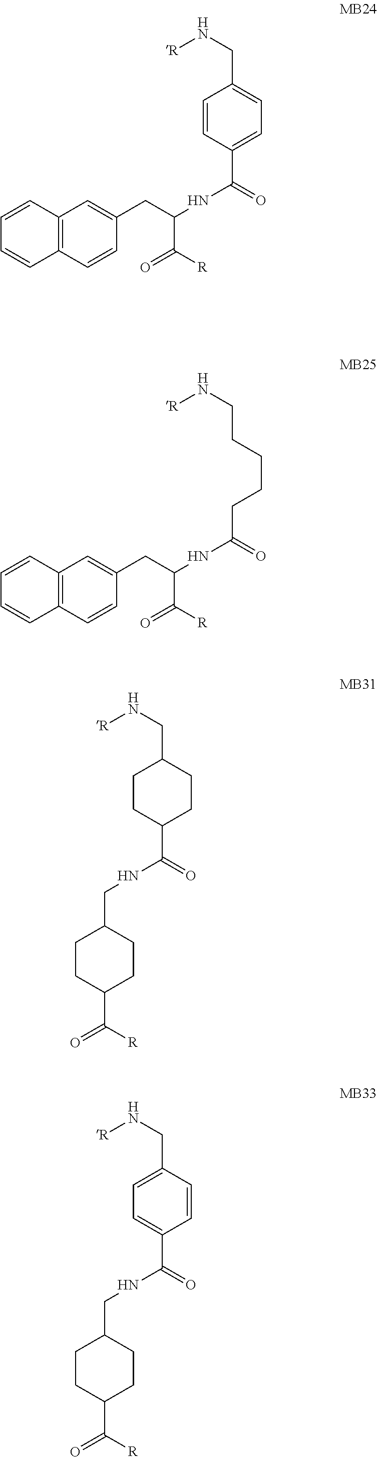

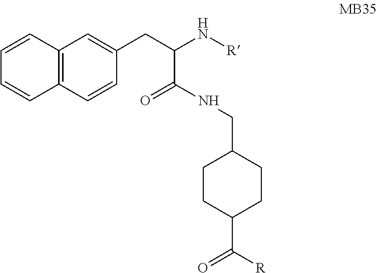

Preferred compounds of the present invention are e.g.
Anderes MB 17 einkleben
   or

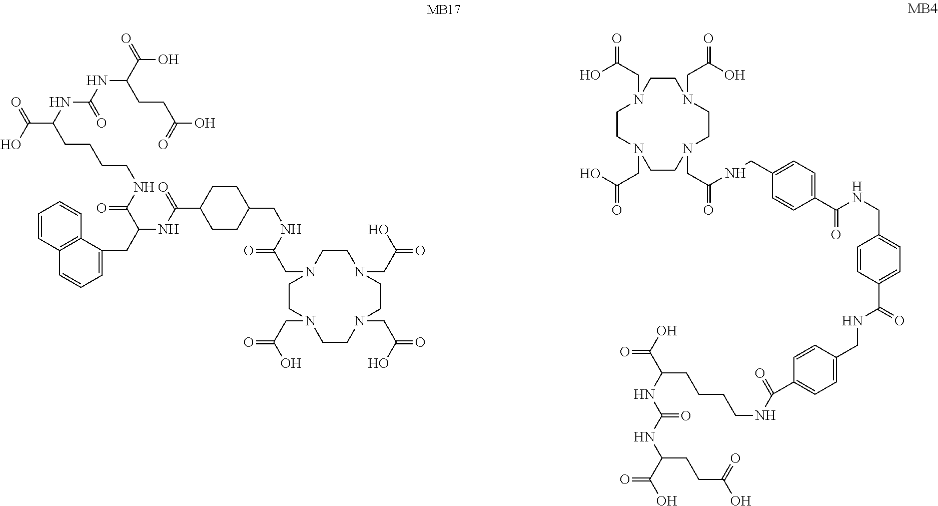 [SMILES: O=C(O)CCC(NC(=O)NC(CCCCNC(=O)C(Cc1cccc2ccccc12)NC(=O)C1CCC(CNC(=O)CN2CCN(CC(=O)O)CCN(CC(=O)O)CCN(CC(=O)O)CC2)CC1)C(=O)O)C(=O)O ; O=C(O)CCC(NC(=O)NC(CCCCNC(=O)c1ccc(CNC(=O)c2ccc(CNC(=O)c3ccc(CNC(=O)CN4CCN(CC(=O)O)CCN(CC(=O)O)CCN(CC(=O)O)CC4)cc3)cc2)cc1)C(=O)O)C(=O)O]

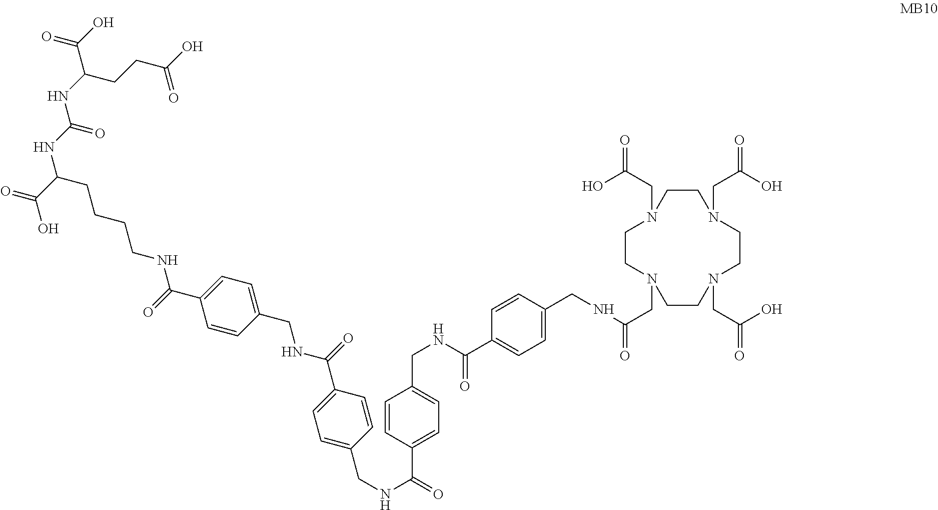 [SMILES: O=C(O)CCC(NC(=O)NC(CCCCNC(=O)c1ccc(CNC(=O)c2ccc(CNC(=O)c3ccc(CNC(=O)c4ccc(CNC(=O)CN5CCN(CC(=O)O)CCN(CC(=O)O)CCN(CC(=O)O)CC5)cc4)cc3)cc2)cc1)C(=O)O)C(=O)O]

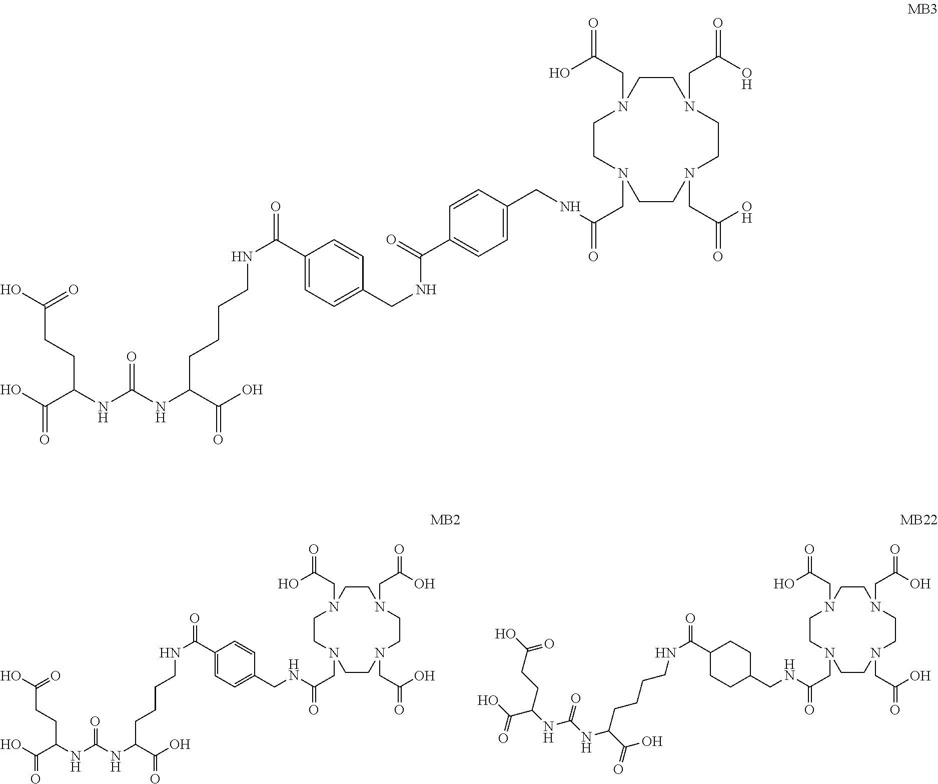 [SMILES: O=C(O)CCC(NC(=O)NC(CCCCNC(=O)c1ccc(CNC(=O)c2ccc(CNC(=O)CN3CCN(CC(=O)O)CCN(CC(=O)O)CCN(CC(=O)O)CC3)cc2)cc1)C(=O)O)C(=O)O ; O=C(O)CCC(NC(=O)NC(CCCCNC(=O)c1ccc(CNC(=O)CN2CCN(CC(=O)O)CCN(CC(=O)O)CCN(CC(=O)O)CC2)cc1)C(=O)O)C(=O)O ; O=C(O)CCC(NC(=O)NC(CCCCNC(=O)C1CCC(CNC(=O)CN2CCN(CC(=O)O)CCN(CC(=O)O)CCN(CC(=O)O)CC2)CC1)C(=O)O)C(=O)O]

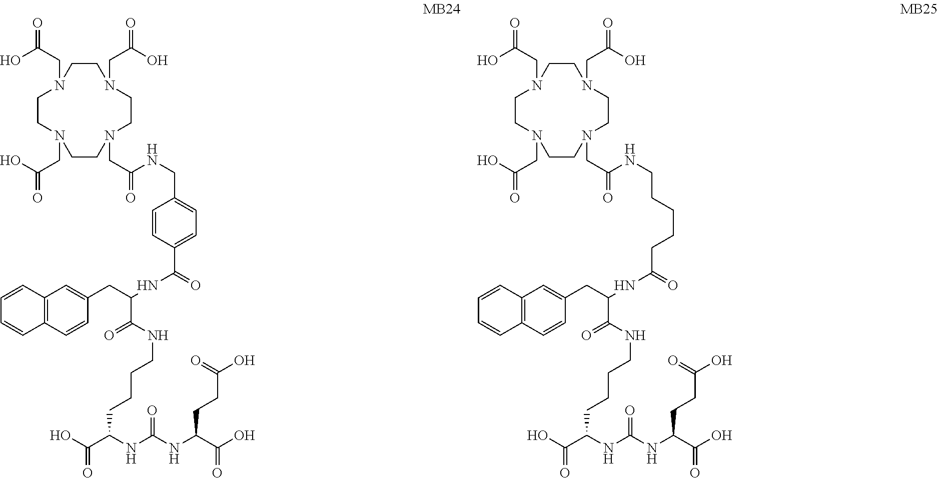 [SMILES: O=C(O)CC[C@H](NC(=O)N[C@@H](CCCCNC(=O)C(Cc1ccc2ccccc2c1)NC(=O)c1ccc(CNC(=O)CN2CCN(CC(=O)O)CCN(CC(=O)O)CCN(CC(=O)O)CC2)cc1)C(=O)O)C(=O)O ; O=C(O)CC[C@H](NC(=O)N[C@@H](CCCCNC(=O)C(Cc1ccc2ccccc2c1)NC(=O)CCCCCNC(=O)CN1CCN(CC(=O)O)CCN(CC(=O)O)CCN(CC(=O)O)CC1)C(=O)O)C(=O)O]

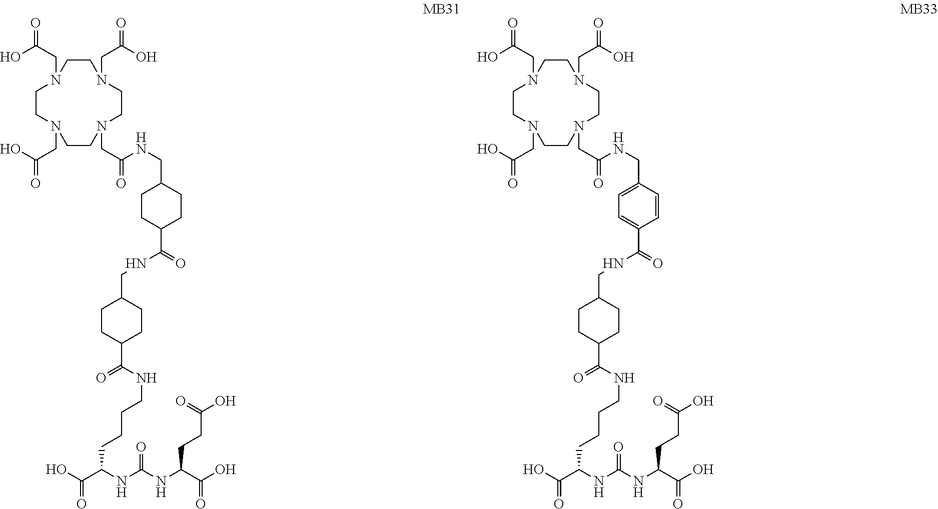 [SMILES: O=C(O)CC[C@H](NC(=O)N[C@@H](CCCCNC(=O)C1CCC(CNC(=O)C2CCC(CNC(=O)CN3CCN(CC(=O)O)CCN(CC(=O)O)CCN(CC(=O)O)CC3)CC2)CC1)C(=O)O)C(=O)O ; O=C(O)CC[C@H](NC(=O)N[C@@H](CCCCNC(=O)C1CCC(CNC(=O)c2ccc(CNC(=O)CN3CCN(CC(=O)O)CCN(CC(=O)O)CCN(CC(=O)O)CC3)cc2)CC1)C(=O)O)C(=O)O]

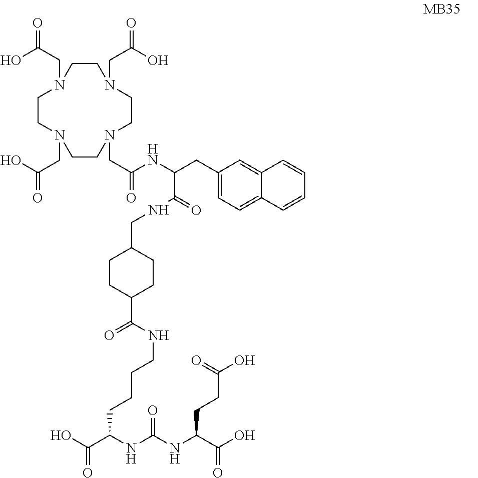 [SMILES: O=C(O)CC[C@H](NC(=O)N[C@@H](CCCCNC(=O)C1CCC(CNC(=O)C(Cc2ccc3ccccc3c2)NC(=O)CN2CCN(CC(=O)O)CCN(CC(=O)O)CCN(CC(=O)O)CC2)CC1)C(=O)O)C(=O)O]

The invention also relates to pharmaceutically acceptable salts of the compounds of general formula (Ia) and/or (Ib). The invention also relates to solvates of the compounds, including the salts as well as the active metabolites thereof and, where appropriate, the tautomers thereof according to general formula (Ia) and/or (Ib) including prodrug formulations.

A “pharmaceutically acceptable salt” is a pharmaceutically acceptable, organic or inorganic acid or base salt of a compound of the invention. Representative pharmaceutically acceptable salts include, e.g., alkali metal salts, alkali earth salts, ammonium salts, water-soluble and water-insoluble salts, such as the acetate, carbonate, chloride, gluconate, glutamate, lactate, laurate, malate or tartrate.

The term “prodrug” refers to a precursor of a drug that is a compound which upon administration to a patient, must undergo chemical conversion by metabolic processes before becoming an active pharmacological agent. Illustrative prodrugs of compounds in accordance with Formula (Ia) and/or (Ib) are esters and amides, preferably alkyl esters of fatty acid esters. Prodrug formulations here may comprise all substances which are formed by simple transformation including hydrolysis, oxidation or reduction either enzymatically, metabolically or in any other way. A suitable prodrug contains e.g. a substance of general formula (Ia) and/or (Ib) bound via an enzymatically cleavable linker (e.g. carbamate, phosphate, N-glycoside or a disulfide group) to a dissolution-improving substance (e.g. tetraethylene glycol, saccharides, formic acids or glucuronic acid, etc.). Such a prodrug of a compound according to the invention can be applied to a patient, and this prodrug can be transformed into a substance of general formula (Ia) and/or (Ib) so as to obtain the desired pharmacological effect.

Some compounds of Formula (Ia) and/or (Ib) are encompassed in form of the racemates, their enantiomers and optionally in form of their diastereomers and all possible mixtures thereof.

According to the invention all chiral C-atoms shall have D- and/or L-configuration; also combinations within one compound shall be possible, i.e. some of the chiral C-atoms may be D- and others may be L-configuration.

The obtained compounds can be optionally separated by known methods (e.g. Allinger, N. L. und Elliel E. L. in “Topics in Stereochemistry” Vol. 6, Wiley Interscience, 1971) in their enantiomers and/or diasteromers. One possible method of enantiomeric separation is the use of chromatography.

The invention also relates to pharmaceutical preparations which contain a therapeutically effective amount of the active ingredients (compound according to the invention of formula (Ia) or (Ib) together with organic or inorganic solid or liquid, pharmaceutically acceptable carriers which are suited for the intended administration and which interact with the active ingredients without drawbacks.

The phrase “pharmaceutically acceptable” is employed herein to refer to those compounds, material, compositions, and/or dosage forms which are, within the scope of sound medical judgment, suitable for use in contact with the tissues of a patient without excessive toxicity, irritation, allergic response, or other problem or complication, commensurate with a reasonable benefit/risk ratio.

A “patient” includes an animal, such as a human, monkey, cow, horse, cat or dog. The animal can be a mammal such as a non-primate and a primate (e.g., monkey and human). In one embodiment, a patient is a human being.

In general, the Formula (Ia) or (Ib) compound or pharmaceutical compositions thereof, may be administered orally or via a parenteral route, usually injection or infusion.

A “parenteral administration route” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticluare, subcapsular, subarachnoid, intraspinal and intrasternal injection and infusion.

The dosage of the compounds according to the invention is determined by the physician on the basis of the patient-specific parameters, such as age, weight, sex, severity of the disease, etc. The dosage is preferably from 0.00001 mg/kg to 100 mg/kg body weight, preferably from 0.001 to 50 mg/kg body weight and most preferably from 0.01 to 10 mg/kg body weight.

Corresponding to the kind of administration, the medicament is suitably formulated, e.g. in the form of solutions or suspensions, simple tablets or dragees, hard or soft gelatine capsules, suppositories, ovules, preparations for injection, which are prepared according to common galenic methods.

The compounds according to the invention can be formulated, where appropriate, together with further active substances and with excipients and carriers common in pharmaceutical compositions, e.g.—depending on the preparation to be produced—talcum, gum arabic, lactose, starch, magnesium stearate, cocoa butter, aqueous and non-aqueous carriers, fatty bodies of animal or vegetable origin, paraffin derivatives, glycols (in particular polyethylene glycol), various plasticizers, dispersants or emulsifiers, pharmaceutically compatible gases (e.g. air, oxygen, carbon dioxide, etc.), preservatives.

In order to produce liquid preparations, additives, such as sodium chloride solution, ethanol, sorbitol, glycerine, olive oil, almond oil, propylene glycol or ethylene glycol, can be used.

When solutions for infusion or injection are used, they are preferably aqueous solutions or suspensions, it being possible to produce them prior to use, e.g from lyophilized preparations which contain the active substance as such or together with a carrier, such as mannitol, lactose, glucose, albumin and the like. The ready made solutions are sterilized and, where appropriate, mixed with excipients, e.g. with preservatives, stabilizers, emulsifiers, solubilizers, buffers and/or salts for regulating the osmotic pressure. The sterilization can be obtained by sterile filtration using filters having a small pore size according to which the composition can be lyophilized, where appropriate. Small amounts of antibiotics can also be added to ensure the maintenance of sterility.

The phrases “effective amount” or “therapeutically-effective amount” as used herein means that amount of a compound, material, or composition which may comprise a compound of the invention, or other active ingredient which is effective for producing some desired therapeutic effect in at least a sub-population of cells in an animal at a reasonable benefit/risk ratio applicable to any medical treatment. A therapeutically effective amount with respect to a compound of the invention means that amount of therapeutic agent alone, or in combination with other therapies, that provides a therapeutic benefit in the treatment of prevention of a disease. Used in connection with a compound of the invention, the term can encompass an amount that improves overall therapy, reduces or avoids symptoms or causes of disease, or enhances the therapeutic efficacy of or synergies with another therapeutic agent.

As used herein, the terms “treating” or “treatment” is intended to encompass also diagnosis, prophylaxis, prevention, therapy and cure.

The terms “prevent”, “preventing,” and “prevention” refer to the prevention of the onset, recurrence, or spread of the disease in a patient resulting from the administration of a prophylactic or therapeutic agent.

Depending on whether the inventive Formula (Ia) and/or (Ib) compounds are to be used as radio-imaging agents or radio-pharmaceuticals different radionuclides are complexed to the chelator. Illustrative radionuclides include, for example, ⁸⁹Zr, ⁴⁴Sc, ¹¹¹In, ⁹⁰Y, ⁶⁶Ga, ⁶⁷Ga, ⁶⁸Ga, ¹⁷⁷Lu, ^{99m}Tc, ⁶¹Cu, ⁶²Cu, ⁶⁴Cu, ⁶⁷Cu, ¹⁴⁹Tb, ¹⁵²Tb, ¹⁵⁵Tb, ¹⁶¹Tb, ¹⁵³Gd, ¹⁵⁵Gd, ¹⁵⁷Gd, ²¹³Bi, ²²⁵Ac, ²³⁰U, ²²³Ra, ¹⁶⁵Er and Fe. According to one aspect of this invention, the radionuclide is ¹¹¹In, ⁹⁰Y, ⁶⁸Ga, ⁶⁴Cu, ¹⁵³Gd, ¹⁵⁵Gd, ²¹³Bi, ²²⁵Ac, Fe, or ¹⁷⁷Lu.

As noted above, complexes of the compounds according Formula (Ia) or (Ib) may contain one or more radionuclides which are suitable for use as radio-imaging agents or as therapeutics for the treatment of rapidly proliferating cells, for example, PSMA expressing prostate cancer cells. According to the present invention they are called “metal complexes” or “radiopharmaceuticals”.

Preferred imaging methods are positron emission tomography (PET) or single photon emission computed tomography (SPECT).

Accordingly, in one embodiment, a pharmaceutical composition is provided including a complex that includes a radionuclide and a compound of Formula (Ia) or Formula (Ib), a salt, solvate, stereoisomer, or tautomer thereof, and a pharmaceutically acceptable carrier.

According to another aspect, a pharmaceutical composition is provided, which is suitable for in vivo imaging and radiotherapy. Suitable pharmaceutical compositions may contain a radio imaging agent, or a radiotherapeutic agent that has a radionuclide either as an element, i.e. radioactive iodine, or a radioactive metal chelate complex of the compound of Formula (Ia) and/or (Ib) in an amount sufficient for imaging, together with a pharmaceutically acceptable radiological vehicle. The radiological vehicle should be suitable for injection or aspiration, such as human serum albumin; aqueous buffer solutions, e.g., tris(hydromethyl) aminomethane (and its salts), phosphate, citrate, bicarbonate, etc; sterile water physiological saline; and balanced ionic solutions containing chloride and or dicarbonate salts or normal blood plasma cautions such as calcium potassium, sodium and magnesium.

The concentration of the imaging agent or the therapeutic agent in the radiological vehicle should be sufficient to provide satisfactory imaging. For example, when using an aqueous solution, the dosage is about 1.0 to 100 millicuries. The actual dose administered to a patient for imaging or therapeutic purposes, however, is determined by the physician administering treatment. The imaging agent or therapeutic agent should be administered so as to remain in the patient for about 1 hour to 10 days, although both longer and shorter time periods are acceptable. Therefore, convenient ampoules containing 1 to 10 mL of aqueous solution may be prepared.

Imaging may be carried out in the normal manner, for example by injecting a sufficient amount of the imaging composition to provide adequate imaging and then scanning with a suitable imaging or scanning machine, such as a tomograph or gamma camera. In certain embodiments, a method of imaging a region in a patient includes the steps of: (i) administering to a patient a diagnostically effective amount of a compound complexed with a radionuclide; exposing a region of the patient to the scanning device; and (ii) obtaining an image of the region of the patient. In certain embodiments of the region imaged is the head or thorax. In other embodiments, the compounds and complexes of Formula I (a) and/or (Ib) target the PSMA protein.

Thus, in some embodiments, a method of imaging tissue such as spleen tissue, kidney tissue, or PSMA-expressing tumor tissue is provided including contacting the tissue with a complex synthesized by contacting a radionuclide and a Formula (Ia) and/or Formula (Ib) compound.

The amount of the compound of the present invention, or a formulation which may comprise a complex of a metal and a compound according to Formula (Ia) and/or (Ib), or its salt, solvate, stereoisomer, or tautomer that is administered to a patient depends on several physiological factors that are routinely used by the physician, including the nature of imaging to be carried out, tissue to be targeted for imaging or therapy and the body weight and medical history of the patient to be imaged or treated using a radiopharmaceutical.

Accordingly in another aspect, the invention provides a method for treating a patient by administering to a patient a therapeutically effective amount of a Formula (Ia) and/or (Ib) compound complexed to a radionuclide, or a pharmaceutically acceptable salt or solvate of the complex to treat a patient suffering from a cell proliferative disease or disorder. Specifically, the cell proliferative disease or disorder to be treated or imaged using a compound, pharmaceutical composition or radiopharmaceutical in accordance with this invention is a cancer, for example, prostate cancer and/or prostate cancer metastasis in e.g. lung, liver, kidney, bones, brain, spinal cord, bladder, etc.

The synthesis of the compounds of the present invention is described in detail in the example section. An overview of the synthesis is exemplified in Scheme 2 concerning DOTA conjugated-PSMA inhibitors. However, a person skilled in the art would be able to modify the reactions e.g. by using another chelator. Thus, this scheme shall not be understood to limit the compounds of the present invention to the DOTA chelator only.

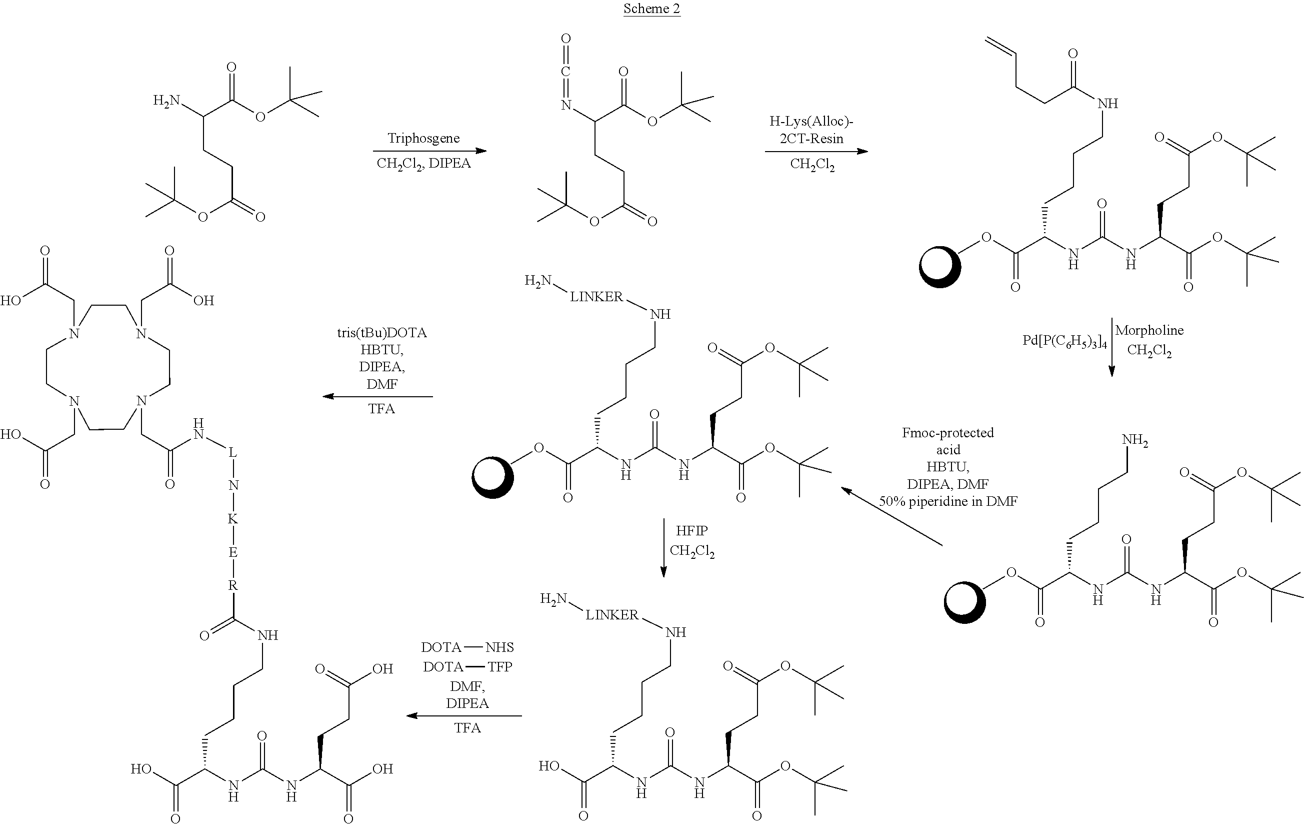

The synthesized compounds are chemically characterized by RP-HPLC, MS, and/or NMR.

The novel chelator-conjugated imaging agents with structural modifications in the linker region have improved tumor targeting properties and pharmacokinetics. The pharmacophore presents three carboxylic group able to interact with the respective side chains of PSMA and an oxygen as part of zinc complexation in the active center. Besides these obligatory interactions, the inventors were able to optimize the lipophilic interactions in the linker region.

The preclinical evaluation includes in vitro assays (affinity, internalization) and in vivo experiments (μPET screening and organ distribution).

The compounds of the present invention are better than known reference compounds with regard to kidney clearance and enrichment in the tumor. The binding affinity of PSMA inhibitors of the present invention can be influenced by linker modifications. Two cyclic motives and at least one aromatic moiety in the linker region of the substance seem to be preferable and resulted in the high affinity compounds MB4 and MB17. In this regard, a very promising compound is MB17.

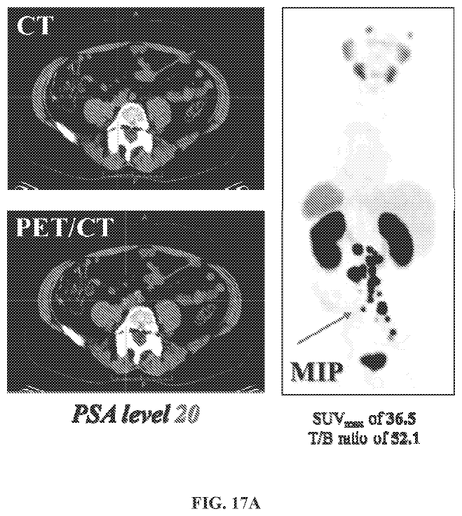

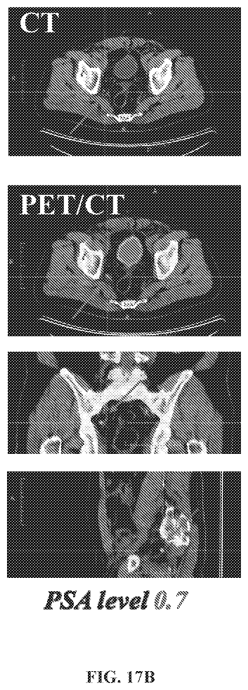

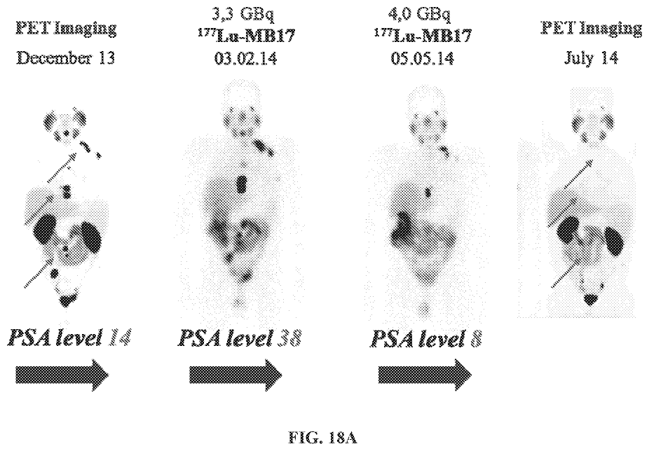

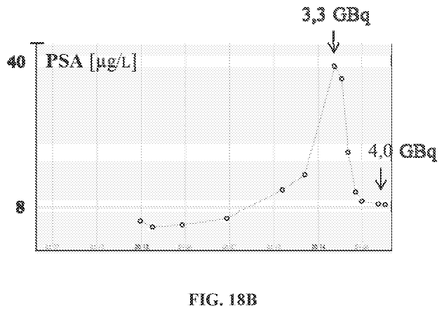

Thus, the compounds of the present invention represent novel PSMA-targeting probes with optimal characteristics which was also confirmed by organ distribution and small animal PET imaging. The compounds of the present invention show a high PSMA-specific tumor uptake. In addition, they are characterized by an early enrichment in the bladder and also the maximum kidney uptake. With regard to therapeutic use, this gives clear clinical advantages for the compounds of the present invention compared to other PSMA-inhibitors. In the PET diagrams the compounds of the present invention, in particular MB17, show a rapid background clearance as well as a substantial reduction of the enrichment in the kidney after 2 hours while it is further accumulated and retained in the PSMA-expressing tumor. Also first in vivo treatments with MB 17 showed promising data (c.f. FIGS. 17 and 18).

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

The below example explains the invention in more detail but are not construed to limit the invention in any way to the exemplified embodiments only.

### EXAMPLES

### Example 1: Synthesis of DOTA-Conjugated Inhibitors

The DOTA conjugated-PSMA inhibitors are synthesized via solid-phase peptide synthesis (c.f. Scheme 2). In a first step, the isocyanate of the glutamyl moiety was generated in situ by adding a mixture of 3 mmol of bis(tert-butyl)-L-glutamate hydrochloride and 3 mL of N-ethyldiisopropylamine (DIPEA) in 200 mL of dry CH₂Cl₂ to a solution of 1 mmol triphosgene in 10 mL of dry CH₂Cl₂ at 5° C. for 3 h. After the reaction, 0.5 mmol of a resin-immobilized (2-chloro-tritylresin) ε-allyloxycarbonyl protected lysine was added and reacted for 16 h with gentle agitation. The resin was filtered off and the allyloxy-protecting group was removed using 50 mg tetrakis-(triphenyl) palladium and 400 μL morpholine in 4 mL CH₂Cl₂ for 2 h.

The subsequent synthesis of the peptidomimetic PSMA binding motif was performed according to standard Fmoc protocol. The following coupling of the linker part was performed using 2 mmol of the corresponding Fmoc-protected acid, 3.96 mmol of HBTU and 2 mmol of N-ethyl-diisopropylamine in a final volume of 4 mL DMF. After activation with 3.95 eq of HBTU and DIPEA for 2 h, 4 eq of tris(t-bu)-DOTA (Chematech) relative to the resin loading were reacted in a final volume of 3 mL DMF. The product was cleaved from the resin in a 2 ml mixture consisting of trifluoroacetic acid, triisopropylsilane, and water (95:2.5:2.5).

The chelator was also conjugated by using HBTU activated DOTA-NHS ester (CheMatech) or DOTA-TFP ester (Mier W., Hoffend J., Krämer S., Schuhmacher J., Hull W. E., Eisenhut M., Haberkom U., Bioconjugate Chem 2005, 16:237-240).

Analysis of the synthesized molecules was performed using reversed-phase high performance liquid chromatography (RP-HPLC; Chromolith RP-18e, 100×4.6 mm, Merck, Darmstadt, Germany) with a linear A-B gradient (0% B to 100% B in 6 min) at a flow rate of 4 mL/min (analysis) or 6 mL/min (purification). Solvent A consisted of 0.1% aqueous TFA and solvent B was 0.1% TFA in CH₃CN. The HPLC system (L6200 A; Merck-Hitachi, Darmstadt, Germany) was equipped with a UV and a gamma detector (Bioscan; Washington, USA). UV absorbance was measured at 214 nm. Mass spectrometry was performed with a MALDI-MS Daltonics Microflex system (Bruker Daltonics, Bremen, Germany).

### Example 2: Radiolabeling

Typically, 1.5 nmol of a synthesized compound of Example 1 (dissolved in 0.1 M HEPES buffer pH 7.5) was added in a volume of 100 μL to a mixture of 10 μL 2.1 M HEPES solution and 40 μL [⁶⁸Ga]Ga³⁺ eluate (40 MBq). The pH of the labeling solution was adjusted to 4.5.

The radiolabeling of the compounds resulted in a radiochemical yield of >97% after 15 minutes at 95° C. and was determined by RP-HPLC and TLC. Subsequent purification was done using Sep-Pak C18 cartridges.

### Example 3: Synthesis of Compounds MB4 and MB17

The isocyanate of the glutamyl moiety was generated in situ by adding a mixture of 3 mmol of bis(tert-butyl) L-glutamate hydrochloride and 1.5 mL of N-ethyldiisopropylamine (DIPEA) in 200 mL of dry CH₂Cl₂ to a solution of 1 mmol triphosgene in 10 mL of dry CH₂Cl₂ at 0° C. over 4 h. After agitation of the reaction mixture for 1 h at 25° C., 0.5 mmol of the resin-immobilized (2-chloro-tritylresin) ε-allyloxycarbonyl protected lysine in 4 mL DCM was added and reacted for 16 h with gentle agitation. The resin was filtered off and the allyloxy-protecting group was removed using 30 mg tetrakis(triphenyl) palladium (0) and 400 μL morpholine in 4 mL CH₂Cl₂ for 3 hours. The following coupling of 3 times 4-(Fmoc-aminomethyl)benzoic acid (in case of MB4) or Fmoc-3-(2-naphthyl)-L-alanine and trans-4-(Fmoc-aminomethyl)cyclohexanecarboxylic acid (in case of MB17), respectively, was performed stepwise using 2 mmol of the Fmoc-protected acid, 1.96 mmol of HBTU and 2 mmol of N-ethyldiisopropylamine in a final volume of 4 mL DMF. After activation with 3.95 eq of HBTU and DIPEA for 2 h, 4 eq of tris(t-bu)-DOTA (Chematech) relative to the resin loading were reacted for 3 h in a final volume of 3 mL DMF. The product was cleaved from the resin in a 2 mL mixture consisting of trifluoroacetic acid, triisopropylsilane, and water (95:2.5:2.5). Purification was performed using RP-HPLC and the purified product was analysed by analytical RP-HPLC and MALDI-MS.

For preparing MB-17D which is the stereoisomer of MB17(L), the synthesis was based on Fmoc-3 (2-naphthyl)-D-alanine. If not stated otherwise, in the present description MB17 means the L-stereoisomer.

### Example 4: Coupling to Various Chelators

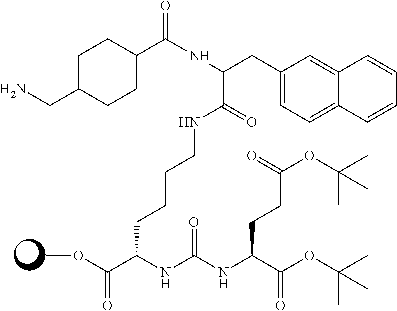 [SMILES: COC(=O)[C@H](CCCCNC(=O)C(Cc1ccc2ccccc2c1)NC(=O)C1CCC(CN)CC1)NC(=O)N[C@@H](CCC(=O)OC(C)(C)C)C(=O)OC(C)(C)C]

The chelators (DOTA, NOTA, NODAGA, DTPA, CHX-DTPA, PCTA, Do3A) were coupled to the MB17 linker by solid phase synthesis. In general, 13 μmol of resin which was coupled with the PSMA binding motif was swollen with DCM in a syringe with a filter. After washing the resin 5× with DMF, it was incubated 2× for 5 min with 20% of piperidine in DMF to deprotect the N-terminus. Another 5× washing with DMF followed.

Between 1.5 and 4 equivalents of the chelator (depending of the chelator), 0.98× n_{chelator} HATU (if needed) and 10 equivalents of DIPEA were dissolved in 500 μl of DMF, the solution was drawn up into the syringe containing the resin and incubated overnight. Next, the resin was washed 5× each with DMF, methanol, DCM and diethyl ether and dried over vacuum.

To check the state of the reaction, test separations were used. This was achieved by washing a small amount of resin with DCM into a filter tip and adding 100 μl of separation solution containing 95% TFA, 2.5% water and 2.5% TIPS. After 30 min of incubation, the solution was pipetted into ice cold diethyl ether and centrifuged. The diethyl ether was decanted and the remaining pellet was dissolved in 35 μl of ACN:H₂O (1:1) and analysed by HPLC (0-100% ACN in water within 5 min) and LC/MS.

If the desired product was obtained, the complete peptide was separated from the resin. The dried resin was incubated with 500 μl of the separation solution (95% TFA, 2.5% H₂O, 2.5% TIPS) for 2 hours. The resulting solution was mixed with ice cold diethyl ether and centrifuged (4000 min⁻¹, 5 min). The supernatant was discarded, new diethyl ether was added and the receptacle was shaken vigorously to resuspend the pellet. Again, the solution was centrifuged (4000 min⁻¹, 5 min) and the resulting supernatant discarded. The pellet was then vacuum dried and finally resuspended in 1 ml of ACN:H₂O (1:1).

Purification was achieved by preparative HPLC, the peaks were analysed by analytic HPLC (0-100% ACN in water within 5 min) and LC/MS and those containing the product were pooled and lyophilized.

### Example 5: Radiolabelling

### ¹⁷⁷Lu-labelling

¹⁷⁷Lu (approx. 100 MBq) was mixed with 200 μl of 0.4 M sodium acetate buffer containing Chelex (pH=5). 10 μl of a 1 mM solution of the compound in 10% DMSO in water, 2 μl of a saturated solution of ascorbic acid and 40 μl of the solution containing ¹⁷⁷Lu were mixed and heated to 95° C. for 10 min. The labelling was checked by radio-HPLC (0-100% ACN in water within 5 min, Monolith column).

### ⁶⁸Ga-Labelling

For the PET scan CHX-DTPA was labelled with ⁶⁸Ga. 1 ml of ⁶⁸Ga was eluted from a ⁶⁸Ge/⁶⁸Ga generator with 0.6 M HCl. 298 μl NaOAc buffer and 1 μl of a 10 mM solution of CHX-DTPA in DMSO was added and incubated for 5 min. Afterwards the product was purified using a SOLA cartridge. Washing was done with a 0.9% NaCl solution and for elution ethanol was used. The ethanol then was vaporized and the remaining product was dissolved in 100 μl of a 0.9% NaCl solution and 10 μl of phosphate buffer.

### Example 6: Determination of the IC₅₀ Value

A filter plate MultiScreen_{HTS}-DV was incubated at room temperature with 100 μl PBS with 1% BSA per well for 30 min. After removing the PBS/BSA solution 10⁵ LNCaP cells in 50 μl of Opti-MEM were applied to each well. Different concentrations of the compounds (leading to concentrations of 0, 0.5, 1, 2.5, 5, 10, 25, 50, 100, 500, 1000 and 5000 nM in each well) in 300 μl of Opti-MEM were mixed with 3 μl of a 150 nM solution of ¹²⁵I-labeled MIP-1466 in Opti-MEM. 50 μl of the resulting solution were added to each well, each concentration was pipetted in quadruples. Each well now contained the radioactively labelled ligand in a concentration of 0.75 nM and the competitive, not labelled ligand in the concentration mentioned above. The plate was then incubated for 45 min at room temperature on a shaker.

After the incubation, the cells were washed 2× with 100 μl of ice cold PBS and 1× with 200 μl of ice cold PBS. Finally, the filters were collected and the remaining radioactivity was measured with a gamma counter. Each tube was measured for 5 min.

The data measured by the gamma counter were evaluated with Graphpad Prism to achieve an inhibition concentration 50 (IC₅₀) against the radioactively labelled MIP-1095.

| Conjugate | IC₅₀ [nM] |
|---|---|
| MB17-DOTA | 0.13 ± 0.08 |
| MB17-NOTA | 0.14 ± 0.08 |
| MB17-DTPA | 0.12 ± 0.05 |
| MB17-CHX-DTPA | 0.06 ± 0.04 |
| MB17-PCTA | 0.10 ± 0.06 |
| MB17-DO3A | 0.10 ± 0.05 |
| MB17-NODAGA | 0.09 ± 0.08 |

### Example 7: μPET-Imaging Using CHX-DTPA-MB17

Before injection into the mouse, the solution containing the purified ⁶⁸Ga-CHX-DTPA-coupled PSMA inhibitor was sterile-filtered. 100 μl of this solution was taken up into a syringe and then injected into a BALB/c nude mouse LNCaP xenograft, intravenously into the tail vein. The PET scan was recorded for 140 min with a Siemens Inveon PET (FIG. 15)

### Example 8: Determination of the Competitive Binding Affinity

In order to compare the series of novel compounds the competitive binding affinity and the specific internalization was analyzed using the PSMA expressing cell line LNCaP. To determine specific cellular uptake, cells were blocked with 2-(phosphonomethyl)-pentanedioic acid (PMPA) The inhibition potency was also investigated by the enzyme-based NAALADase assay.

### Cell Culture

For binding studies and in vivo experiments LNCaP cells (metastatic lesion of human prostatic adenocarcinoma, ATCC CRL-1740) were cultured in RPMI medium supplemented with 10% fetal calf serum and Glutamax (PAA, Austria). During cell culture, cells were grown at 37° C. in an incubator with humidified air, equilibrated with 5% CO₂. The cells were harvested using trypsin-ethylenediaminetetraacetic acid (trypsin-EDTA; 0.25% trypsin, 0.02% EDTA, all from PAA, Austria) and washed with PBS.

### Cell Binding and Internalization

The competitive cell binding assay and internalization experiments were performed as described previously (Eder et al. 2012). Briefly, the respective cells (10⁵ per well) were incubated with the radioligand (68Ga-labeled [Glu-urea-Lys (Ahx)]2-HBED-CC (Schafer et al, 2012) in the presence of 12 different concentrations of analyte (0-5000 nM, 100 L/well). After incubation, washing was carried out using a multiscreen vacuum manifold (Millipore, Billerica, MA). Cell-bound radioactivity was measured using a gamma counter (Packard Cobra II, GMI, Minnesota, USA). The 50% inhibitory concentration (IC50) was calculated by fitting the data using a nonlinear regression algorithm (GraphPad Software). Experiments were performed three times.

To determine the specific cell uptake and internalization, 10⁵ cells were seeded in poly-L-lysine coated 24-well cell culture plates 24 h before incubation. After washing, the cells were incubated with 25 nM of the radiolabeled compounds for 45 min at 37° C. and at 4° C., respectively. Specific cellular uptake was determined by competitive blocking with 2-(phosphonomethyl) pentanedioic acid (500 μM final concentration, PMPA, Axxora, Loerrach, Germany). Cellular uptake was terminated by washing 4 times with 1 mL of ice-cold PBS. Cells were subsequently incubated twice with 0.5 mL glycine-HCl in PBS (50 mM, pH=2.8) for 5 min to remove the surface-bound fraction. The cells were washed with 1 mL of ice-cold PBS and lysed using 0.3 N NaOH (0.5 mL). The surface-bound and the internalized fractions were measured in a gamma counter. The cell uptake was calculated as percent of the initially added radioactivity bound to 10⁶ cells [% ID/10⁶ cells].

### Naaladase Assay

Recombinant human PSMA (rhPSMA, R&D systems, Wiesbaden, Germany) was diluted in assay buffer (50 mM HEPES, 0.1 M NaCl, pH 7.5) to 0.4 μg/mL. The substrate Ac-Asp-Glu (Sigma, Taufkirchen, Germany, 40 μM final concentration) was mixed with natGa labeled analyte at concentrations ranging from 0.05 nM to 1000 nM in a final volume of 125 μL assay buffer. The mixtures were combined with 125 μL of the rhPSMA solution (0.4 μg/mL) and incubated for one hour at 37° C. The reaction was stopped by heating at 95° C. for 5 minutes. 250 μL of a 15 mM solution of ortho-phthaldialdehyde (Sigma, Taufkirchen, Germany) was added to all vials and incubated for 10 minutes at ambient temperature. Finally, 200 μL of the reaction solutions were loaded onto a F16 Black Maxisorp Plate (Nunc, Langenselbold, Germany) and read at excitation and emission wavelengths of 330 nm and 450 nm, respectively, using a microplate reader (DTX-880, Beckman Coulter, Krefeld, Germany). The data were analyzed by a one site-total binding regression algorithm of GraphPad (GraphPad Software, California, USA).

### Biodistribution

7- to 8-week-old male BALB/c nu/nu mice (Charles River Laboratories) were subcutaneously inoculated into the right trunk with 5×10⁶ cells of LNCaP (in 50% Matrigel; Becton Dickinson, Heidelberg, Germany). The tumors were allowed to grow until approximately 1 cm³ in size. The radiolabeled compounds were injected into the tail vein (approx. 1 MBq per mouse; 0.06 nmol). At 1 h after injection the animals were sacrificed. Organs of interest were dissected, blotted dry, and weighed. The radioactivity was measured using a gamma counter and calculated as % ID/g.

### MicroPET

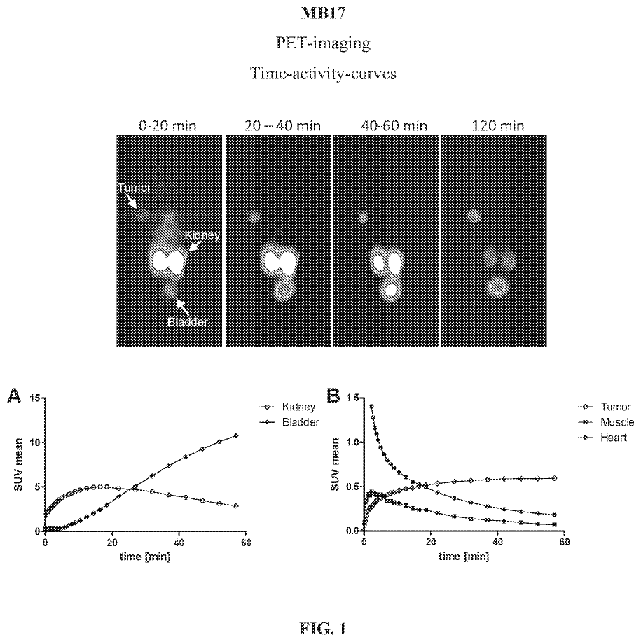

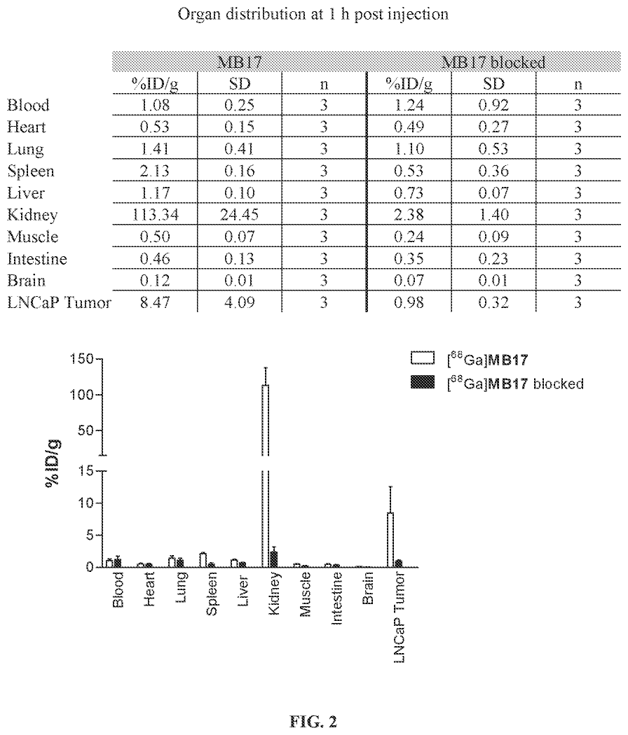

For the microPET studies, 10-25 MBq of the radiolabeled compounds in a volume of 0.15 ml (˜ 0.5 nmol) were injected via a lateral tail vein into mice bearing LNCaP tumor xenografts. The anesthetized animals (2% sevoflurane, Abbott, Wiesbaden, Germany) were placed in prone position into the Inveon small animal PET scanner (Siemens, Knoxville, Tenn, USA) to perform dynamic microPET scans and 20 min-static scans; c.f. FIG. 1, 3, 5-14

TABLE A

| | IC₅₀ | Internalization |
|---|---|---|
| Substance | [nM] | [% IA/10⁶ cells] |
| MB2 | 2.75 ± 0.82 | 8.78 ± 3.96 for Ga-68 |
| | | 5.22 ± 0.67 for Lu-177 |
| MB3 | 10.51 ± 6.06 | 3.65 ± 1.32 for Lu-177 |
| MB4 | 0.74 ± 0.50 | 14.18 ± 0.98 for Ga-68 |
| | | 14.25 ± 4.61 for Lu-177 |
| MB10 | 8.67 ± 1.58 | 6.96 ± 3.90 for Lu-177 |
| MB17 | 0.13 ± 0.08 | 17.02 ± 4.36 for Ga-68 |
| | | 17.51 ± 3.99 for Lu-177 |
| MB17.D | 12.41 ± 5.10 | 2.60 ± 0.14 for Lu-177 |
| MB22 | 52.80 | 1.15 ± 0.19 for Lu-177 |
| MB24 | 3.33 | 7.26 ± 2.76 for Lu-177 |
| MB25 | 6.64 | 3.91 ± 0.54 forLu-177 |
| MB31 | 91.80 | 0.53 ± 0.48 for Lu-177 |
| MB33 | 59.33 | 1.96 ± 0.20 for Lu-177 |
| MB35 | 26.18 | 0.97 ± 0.17 for Lu-177 |

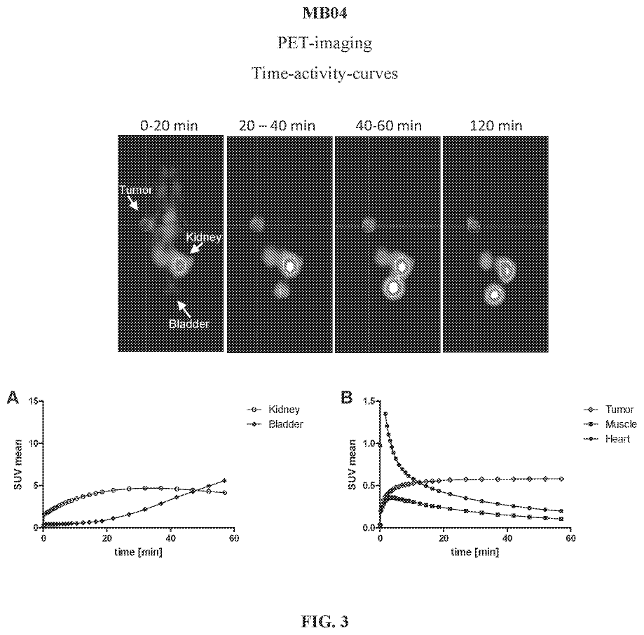

The present example shows that the binding affinity of PSMA inhibitors can be influenced by linker modifications. Two cyclic motives and at least one aromatic moiety in the linker region of the substance seem to be preferable and resulted in the high affinity compounds MB4 and MB17. These novel variants show low nanomolar affinity to LNCap cell line and were specifically internalized at 37° C. up to 48% ID/10⁶ cells. Former studies showed that besides binding affinity the internalization properties of PSMA-targeting probes are highly important and high internalization rates are essential for high in vivo tumor uptake and retention. Thus, MB17 represents a novel PSMA-targeting probe with optimal characteristics which was also confirmed by organ distribution and small animal PET imaging. MB 17 shows a high PSMA-specific tumor uptake (FIG. 2). In addition, dynamic PET imaging of MB17 (FIG. 2) shows an early enrichment in the bladder and also the maximum kidney uptake (highest point in the time-activity-curve) is as early as 15 min after injection of the radiotracer and diminishes substantially already after 20 minutes. With regard to therapeutic use, this gives clear clinical advantages for MB17 compared to other PSMA-inhibitors. In the PET diagrams (FIG. 1) MB17 shows a rapid background clearance as well as a substantial reduction of the enrichment in the kidney after 2 hours while it is further accumulated and retained in the PSMA-expressing tumor.

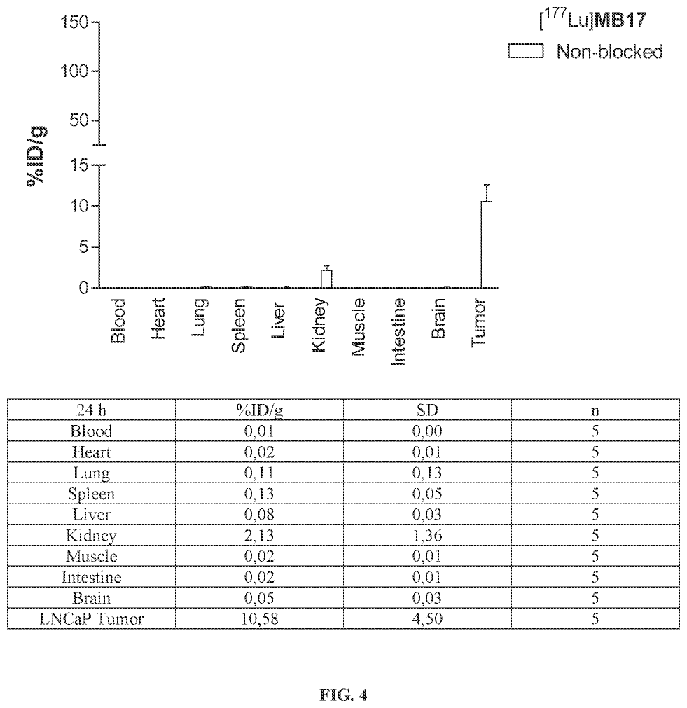

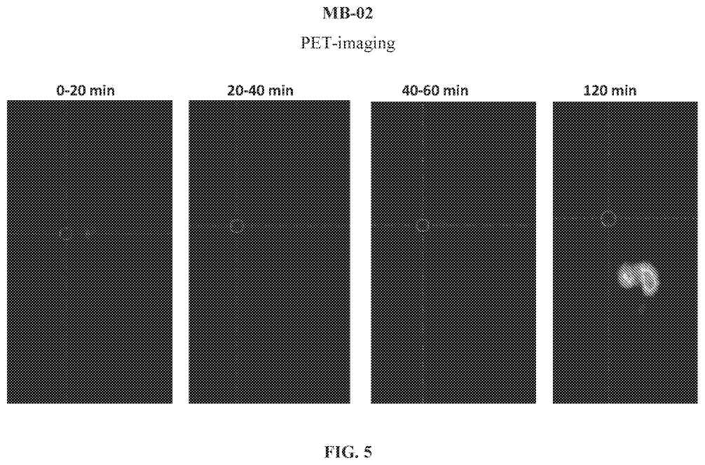

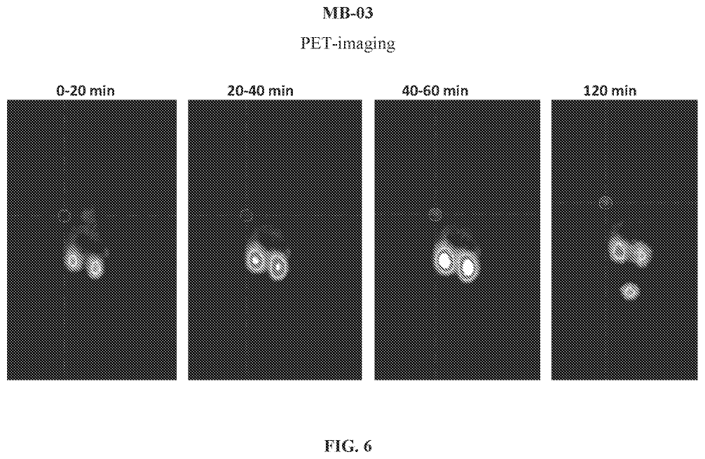

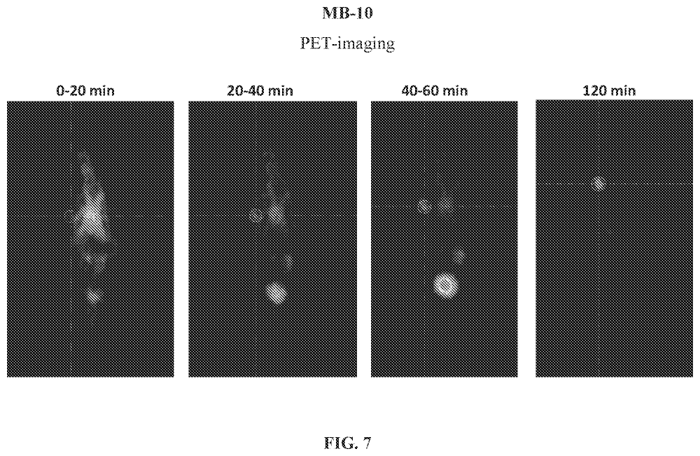

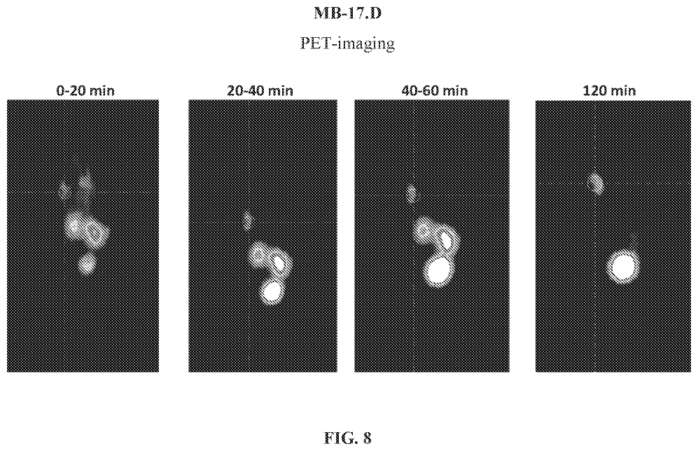

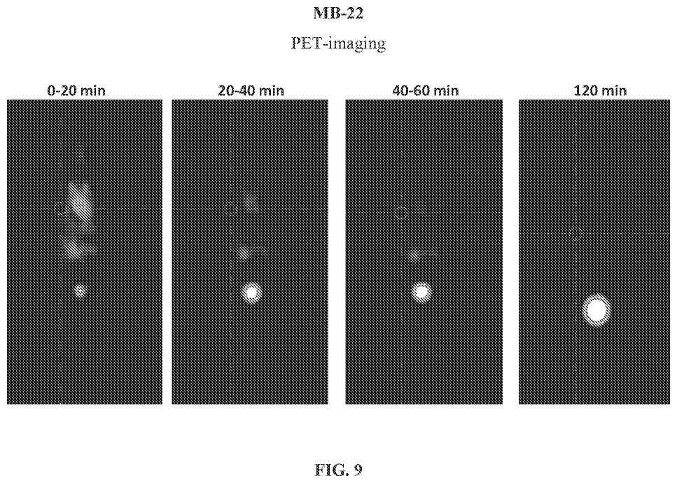

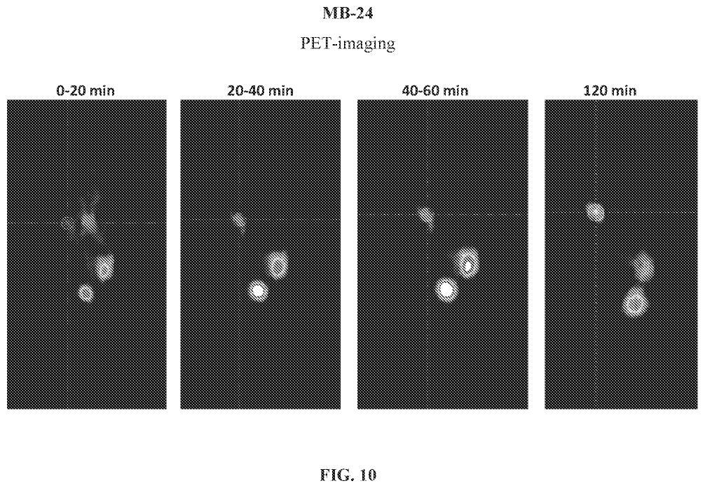

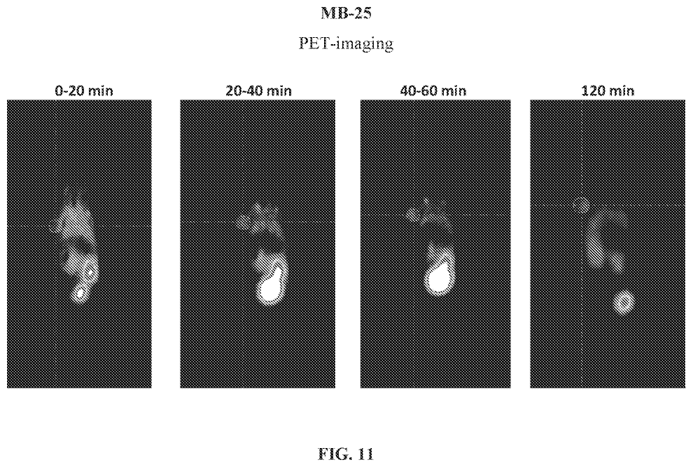

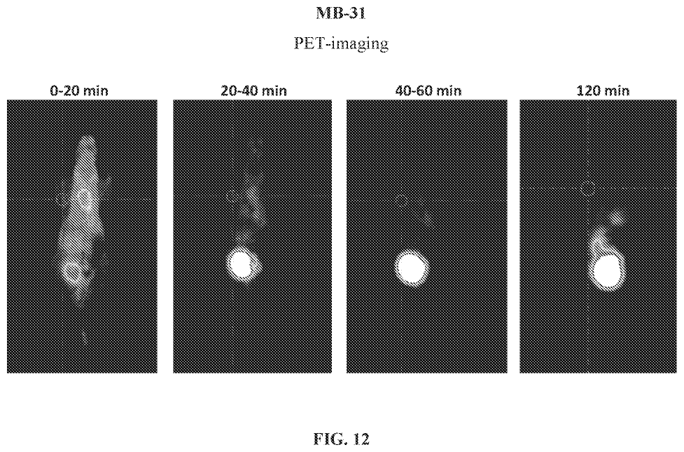

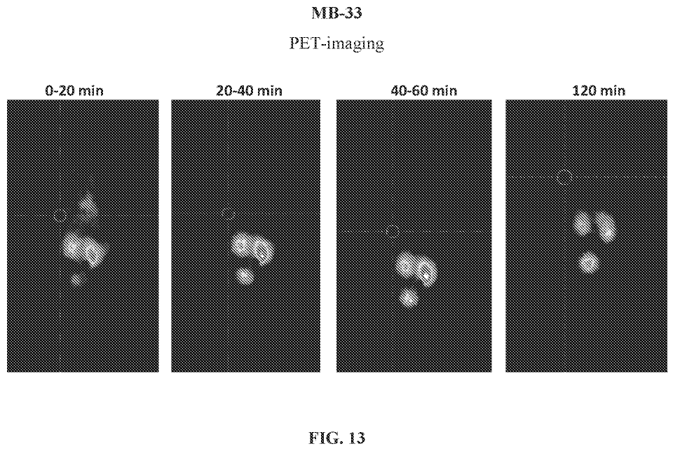

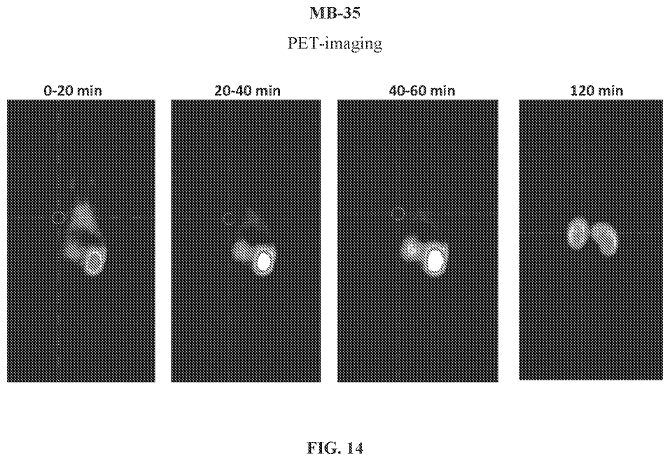

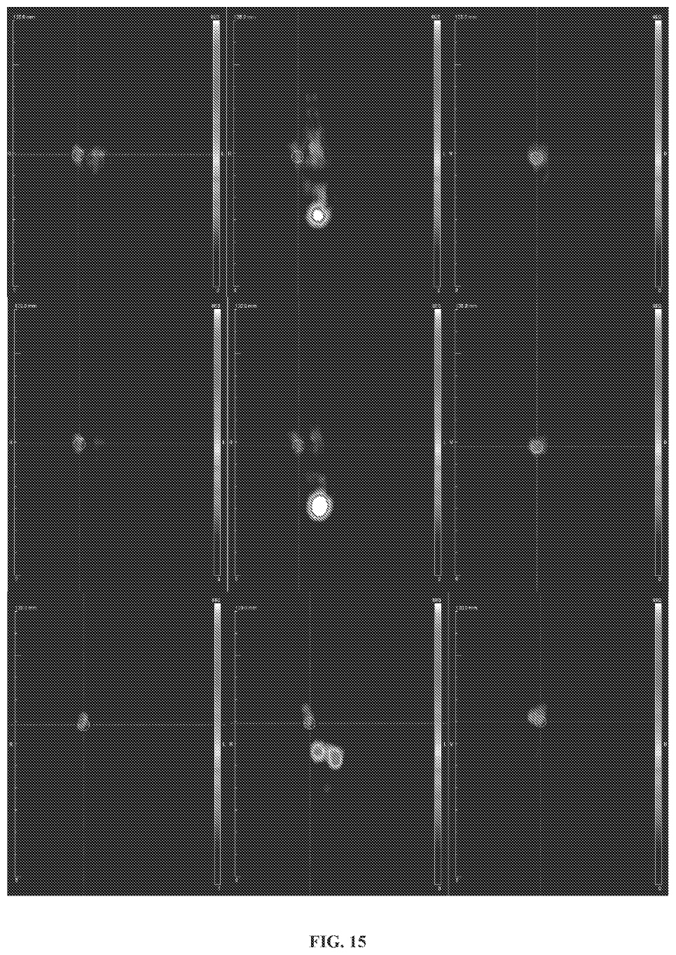

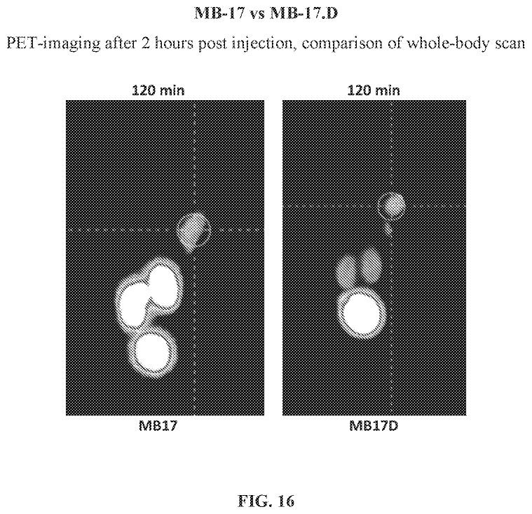

In addition, organ distribution with ¹⁷⁷Lu (FIG. 4) showed that the high initial kidney uptake is nearly completely washed out (2.13±1.36% ID/g) after 24 hours while the tumor uptake remained high and even increased (10.58±4.50% ID/g). Other organs as liver (0.08±0.03% ID/g), lung (0.11±0.13% ID/g) and spleen (0.13±0.05% ID/g) showed very low uptake. The favourable pharmacokinetics led to extremely high tumor-to-background ratios (Tumor/Blood: 1058; Tumor/Muscle: 529) after 24 hours

Table A clearly confirms that the chemical modifications in the linker region of the molecule affect the biological properties, e.g. affinity and internalization efficacy. MB17 and MB4 show the most promising binding properties on cells.

### Example 9: Clinical Data Concerning MB17

PET/CT imaging was performed using the radiotracer MB17 labeled with Ga-68 (c.f FIG. 17)

The ⁶⁸Ge/⁶⁸Ga-generator used for radiopharmaceutical production was purchased from IDB-Holland BV (Baarle-Nassau, The Netherlands). Disposable cassette kits and chemicals including the precursor in GMP-compliant grade used for the radiosynthesis were obtained from ABX advanced biochemical compounds (Radeberg, Germany). An Ultimate 3000 HPLC system (Dionex) (acetonitrile (A), water+0.1% TFA (B); gradient: 0.5 min 95% B, 10.0 min 80% A, flowrate: 2 mL/min) equipped with a Chromolith Performance RP-18e column (100×4.6 mm, Merck) and a NaI radiodetector (Raytest) was used to determine the radiochemical purity. Residual solvents were determined using a 6850 Series gas chromatograph (Agilent Technologies). Endotoxin testing was performed with an Endosafe®-PTS device (Charles River).

2 μg of MB17 were dissolved in 1.5 M acetate buffer pH 4.5 (1 mL) and 1 M ascorbic acid (10 μL) and transferred into the reaction vessel. The ⁶⁸Ge/⁶⁸Ga-generator was eluted with 10 mL of 0.6 M HCl and the eluate diluted with 9 mL of ultrapure water. The mixture was then transferred to a cation exchange cartridge (Macherey-Nagel PS-H+, Size M) and eluted with 5 M NaCl solution (1.2 mL) into the preheated reaction vessel (100° C.). The reaction mixture was heated for 10 minutes. The crude reaction mixture was then removed from the reaction vessel and transferred to a pre-conditioned (10 mL EtOH/10 mL ultrapure water) C18 cartridge (Waters Sep-Pak light). 9 mL ultrapure water was used to rinse the reaction vessel and passed over the C18 cartridge. The C18 cartridge was washed with another 5 mL of ultrapure water. The final product was eluted from the C18 cartridge with 2 mL of EtOH/H₂O (v:v 1:1), sterile filtered (Millipore Cathivex-GV, 0.22 μm) and diluted with 10 mL of phosphate buffered saline (PBS) solution pH 7.4 (according to Eur. Ph. 8.0 (4005000)). The ⁶⁸Ga-MB17 complex solution was applied to patients via an intravenous bolus.

### Example 10: Human Therapy with ¹⁷⁷Lu-Labeled MB17

For therapy, the PSMA ligand MB17 was radiolabeled with Lu-177. ¹⁷⁷LuCl₃ was obtained from Perkin Elmer (4 GBq, NEZ307D, 0.04 M HCl). 80 nmoles of MB17 were dissolved in 400 μL sodium acetate buffer (0.4 M, pH 5) supplemented with 5 μL of 20% ascorbic acid. The solution was transferred to the ¹⁷⁷LuCl₃ and incubated for 10 minutes at 95° C. Finally, 2 mL 0.9% NaCl was added. For quality control, ITLC and radio-HPLC was performed.

The ¹⁷⁷Lu-labeled MB17 was applied to patients via an intravenous bolus (5 ml., slowly within 30 seconds). The intravenous application was accompanied by an infusion of 0.9% NaCl for 4.5 h starting at 0.5 h before injection. Reference is made to FIG. 18. The invention is further described by the following numbered paragraphs:
1. A compound of Formula (Ia) or (Ib):

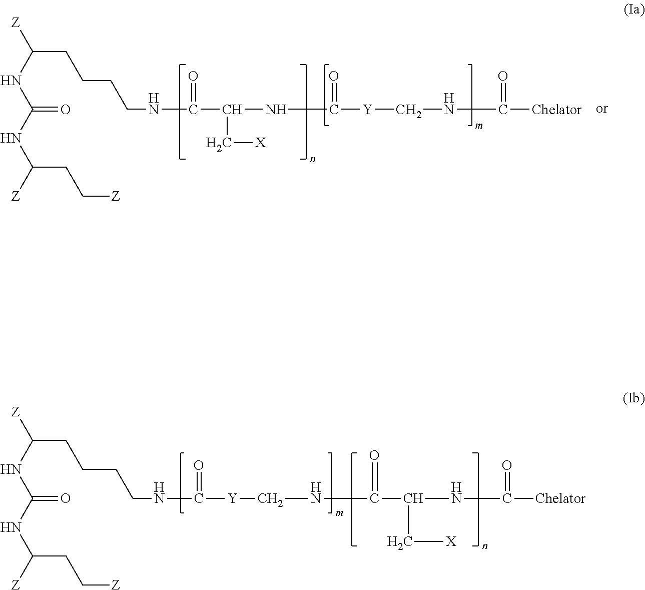

with:

| | |
|---|---|
| n: | 0, 1 |
| m: | 1, 2, 3, 4 |
| Z: | —CO₂H, —SO₂H, —SO₃H, —SO₄H, —PO₂H, —PO₃H, —PO₄H₂ |
| X: | Naphthyl, Phenyl, Biphenyl, Indolyl (=2,3-benzopyrrolyl), Benzothiazolyl |
| Y: | Aryl, Alkylaryl, Cyclopentyl, Cyclohexyl, Cycloheptyl |
| Chelator | 1,4,7,10-tetraazacyclododecane-N,N′,N″,N″′-tetraacetic acid (=DOTA), |
| radical of: | N,N″-bis[2-hydroxy-5-(carboxyethyl)benzyl]ethylenediamine-N,N″- |
| | diacetic acid (HBED-CC), |
| | 1,4,7-triazacyclononane-1,4,7-triacetic acid (NOTA), |
| | 2-(4,7-bis(carboxymethyl)-1,4,7-triazonan-1-yl)pentanedioic acid |
| | (NODAGA), |
| | 2-(4,7,10-tris(carboxymethyl)-1,4,7,10-tetraazacyclododecan-1- |
| | yl)pentanedioic acid |
| | (DOTAGA), |
| | 1,4,7-triazacyclononane phosphinic acid (TRAP), |
| | 1,4,7-triazacyclononane-1-[methyl(2-carboxyethyl)phosphinic acid]-4,7- |
| | bis[methyl(2-hydroxymethyl)phosphinic acid] (NOPO), |
| | 3,6,9,15-tetraazabicyclo[9.3.1]pentadeca-1(15),11,13-triene-3,6,9- |
| | triacetic acid (PCTA), |
| | N′-{5-[Acetyl(hydroxy)amino]pentyl}-N-[5-({4-[(5- |
| | aminopentyl)(hydroxy)amino]-4-oxobutanoyl}amino)pentyl]-N- |
| | hydroxysuccinamide (DFO), |
| | Diethylenetriaminepentaacetic acid (DTPA) |
| | Trans-cyclohexyl-diethylenetriaminepentaacetic acid (CHX-DTPA) |
| | 1-oxa-4,7,10-triazacyclododecane-4,7,10-triacetic acid (oxo-Do3A) |
| | p-isothiocyanatobenzyl-DTPA (SCN-Bz-DTPA) |
| | 1-(p-isothiocyanatobenzyl)-3-methyl-DTPA (1B3M) |
| | 2-(p-isothiocyanatobenzyl)-4-methyl-DTPA (1M3B) |
| | 1-(2)-methyl-4-isocyanatobenzyl-DTPA (MX-DTPA). |

2. The compound of paragraph 1 having the structure R-Linker-R with R=DOTA and R=Glu-Urea-Lys:

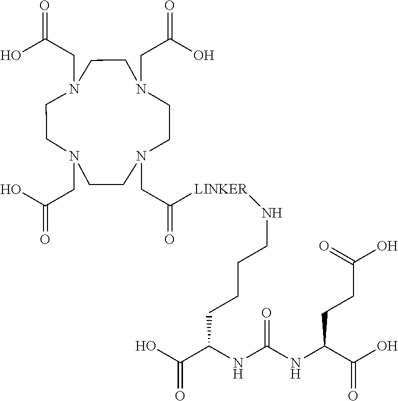

wherein the linker is selected from:

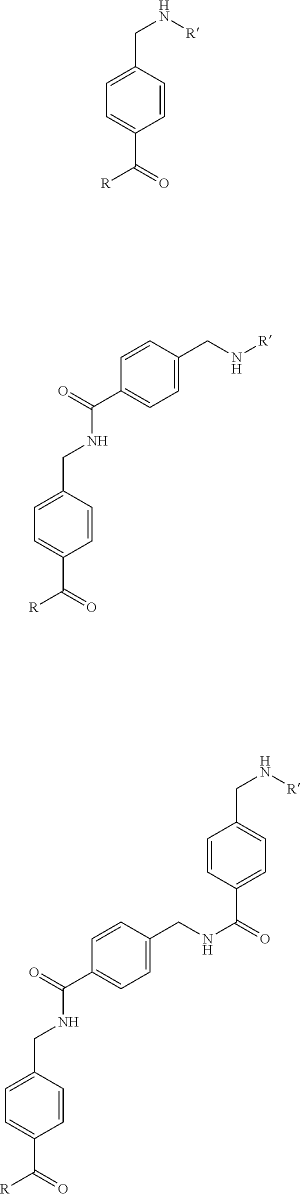

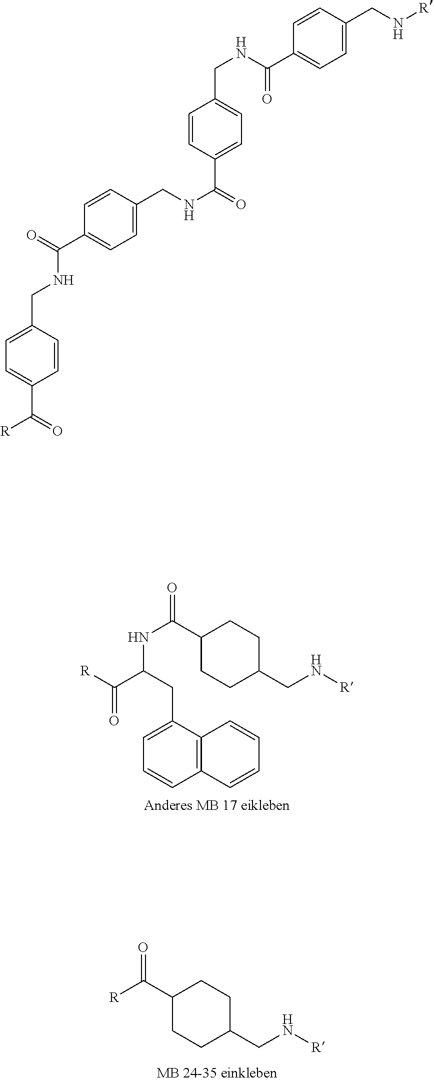

3. The compound of paragraph 1 or 2, selected from the following:

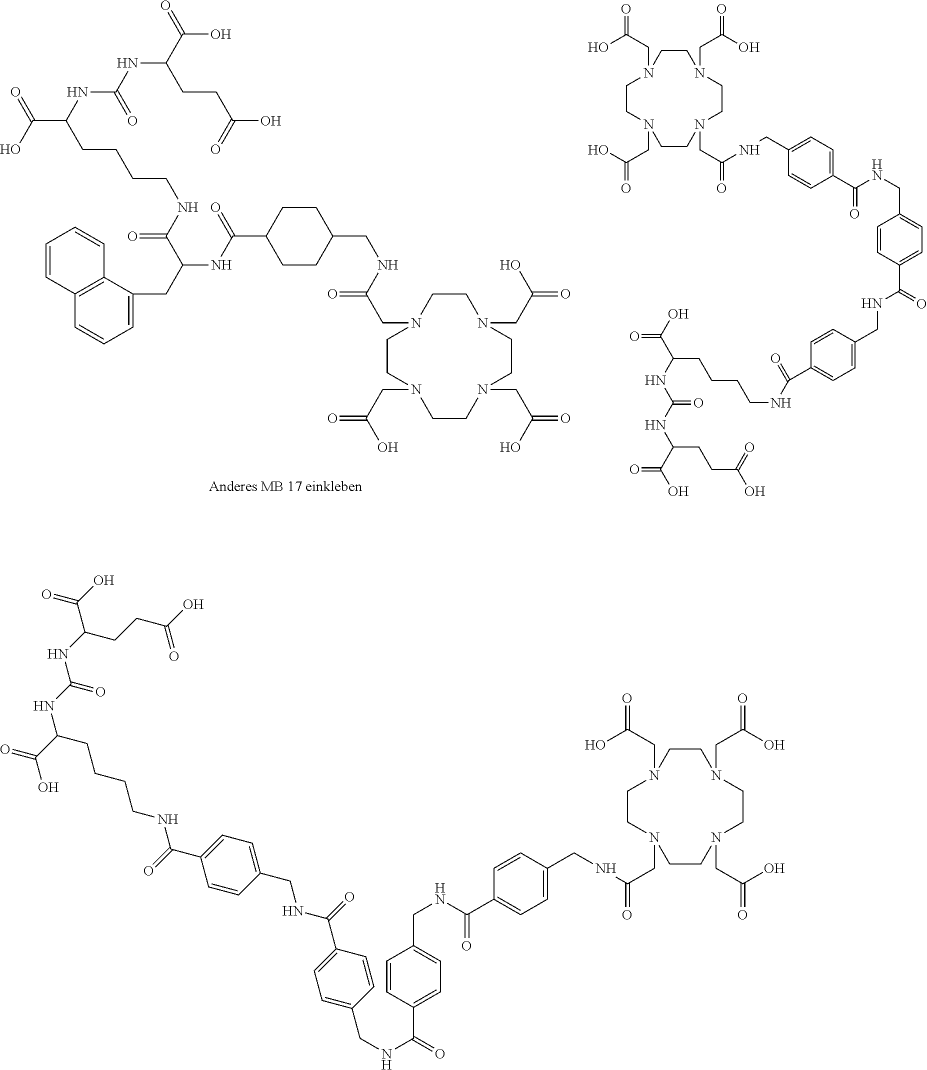 [SMILES: O=C(O)CCC(NC(=O)NC(CCCCNC(=O)C(Cc1cccc2ccccc12)NC(=O)C1CCC(CNC(=O)CN2CCN(CC(=O)O)CCN(CC(=O)O)CCN(CC(=O)O)CC2)CC1)C(=O)O)C(=O)O ; O=C(O)CCC(NC(=O)NC(CCCCNC(=O)c1ccc(CNC(=O)c2ccc(CNC(=O)c3ccc(CNC(=O)CN4CCN(CC(=O)O)CCN(CC(=O)O)CCN(CC(=O)O)CC4)cc3)cc2)cc1)C(=O)O)C(=O)O ; O=C(O)CCC(NC(=O)NC(CCCCNC(=O)c1ccc(CNC(=O)c2ccc(CNC(=O)c3ccc(CNC(=O)c4ccc(CNC(=O)CN5CCN(CC(=O)O)CCN(CC(=O)O)CCN(CC(=O)O)CC5)cc4)cc3)cc2)cc1)C(=O)O)C(=O)O]

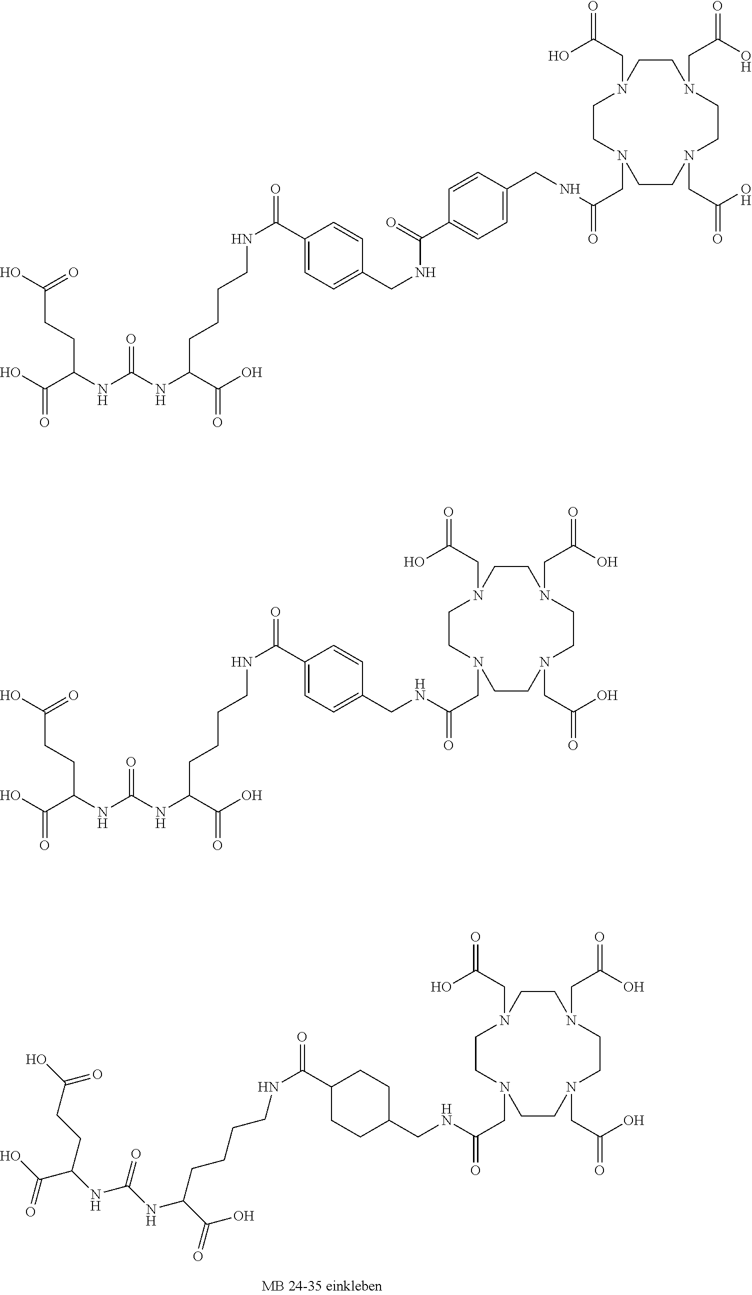 [SMILES: O=C(O)CCC(NC(=O)NC(CCCCNC(=O)c1ccc(CNC(=O)c2ccc(CNC(=O)CN3CCN(CC(=O)O)CCN(CC(=O)O)CCN(CC(=O)O)CC3)cc2)cc1)C(=O)O)C(=O)O ; O=C(O)CCC(NC(=O)NC(CCCCNC(=O)c1ccc(CNC(=O)CN2CCN(CC(=O)O)CCN(CC(=O)O)CCN(CC(=O)O)CC2)cc1)C(=O)O)C(=O)O ; O=C(O)CCC(NC(=O)NC(CCCCNC(=O)C1CCC(CNC(=O)CN2CCN(CC(=O)O)CCN(CC(=O)O)CCN(CC(=O)O)CC2)CC1)C(=O)O)C(=O)O]

4. Use of the compound of any of paragraphs 1 to 3 for the preparation of radiolabeled compounds.
5. A metal complex comprising a radionuclide and a compound of any of paragraphs 1 to 3.
6. The metal complex of paragraph 5, wherein the radionuclide is ¹¹¹In, ⁹⁰Y, ⁶⁸Ga, ¹⁷⁷Lu, ^{99m}Tc, ⁶⁴Cu, ¹⁵³Gd, ¹⁵⁵Gd, ¹⁵⁷Gd, ²¹³Bi, ²²⁵Ac or Fe.
7. A pharmaceutical composition comprising a compound of any of paragraphs 1 to 3 or metal complex of paragraph 5 or 6, or a pharmaceutically acceptable salt, or ester thereof, and a pharmaceutically acceptable carrier.
8. Compound of any of paragraphs 1 to 3 or metal complex of paragraph 5 or 6 for use in a method of imaging in a patient.
9. Compound of any of paragraphs 1 to 3 or metal complex of paragraph 5 or 6 for use in a method of diagnosing prostate cancer and/or metastasis thereof.
10. Compound of any of paragraphs 1 to 3 or metal complex of paragraph 5 or 6 for use in a method of treating prostate cancer and/or metastasis thereof.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1-14. (canceled)

15. A compound of the formula: 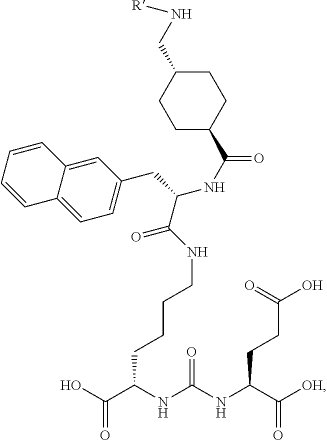
or a salt thereof,
wherein R′ is a chelator of the formula: 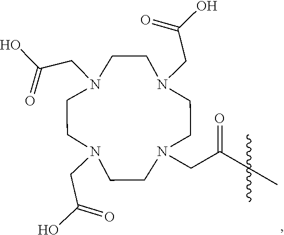 [SMILES: *C(=O)CN1CCN(CC(=O)O)CCN(CC(=O)O)CCN(CC(=O)O)CC1]
and
wherein ¹⁶¹Tb is complexed to the chelator.

16. A composition comprising:
(1) the compound and/or a salt thereof according to claim 15; and
(2) optionally a pharmaceutically acceptable carrier.

17. The composition of claim 16, wherein the composition further comprises an excipient, and wherein the excipient is different from said pharmaceutically acceptable carrier.

18. The composition of claim 16, wherein the composition is a buffered solution.

19. The composition of claim 16, wherein the pharmaceutically acceptable carrier is selected from mannitol, lactose, glucose, albumin, or a combination thereof.

20. A compound of the formula: 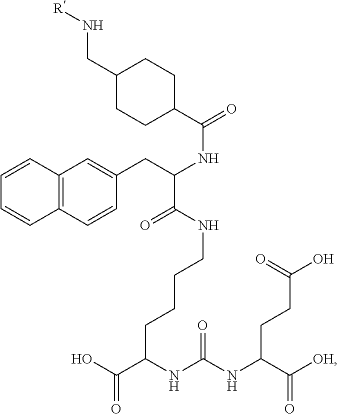
or a salt thereof,
wherein R′ is a chelator of the formula: 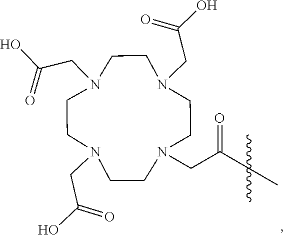 [SMILES: *C(=O)CN1CCN(CC(=O)O)CCN(CC(=O)O)CCN(CC(=O)O)CC1]
and
wherein ¹⁶¹Tb is complexed to the chelator.

21. A composition comprising:
(1) the compound and/or a salt thereof according to claim 20; and
(2) optionally a pharmaceutically acceptable carrier.

22. The composition of claim 21, wherein the composition further comprises an excipient, and wherein the excipient is different from said pharmaceutically acceptable carrier.

23. The composition of claim 21, wherein the composition is a buffered solution.

24. The composition of claim 21, wherein the pharmaceutically acceptable carrier is selected from mannitol, lactose, glucose, and albumin.

25. A compound of the formula: 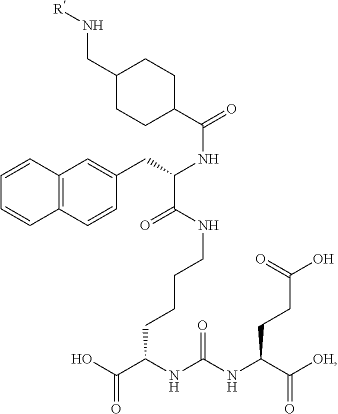
or a salt thereof,
wherein R′ is a chelator of the formula: 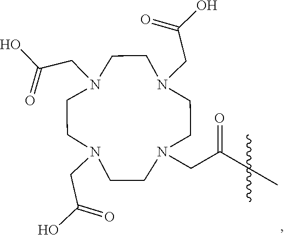 [SMILES: *C(=O)CN1CCN(CC(=O)O)CCN(CC(=O)O)CCN(CC(=O)O)CC1]
and
wherein ¹⁶¹Tb is complexed to the chelator.

26. A composition comprising:
(1) the compound and/or a salt thereof according to claim 25; and
(2) optionally a pharmaceutically acceptable carrier.

27. The composition of claim 26, wherein the composition further comprises an excipient, and wherein the excipient is different from said pharmaceutically acceptable carrier.

28. The composition of claim 26, wherein the composition is a buffered solution.

29. The composition of claim 26, wherein the pharmaceutically acceptable carrier is selected from mannitol, lactose, glucose, albumin, or a combination thereof.
